SEQUENCE LISTING (75 sequences)

SEQ ID NO: 1 - DNA, 10641 bp, Artificial sequence
  Synthetic polynucleotide
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgctttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 2 - DNA, 16208 bp, Artificial sequence
  Synthetic polynucleotide
attcctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaacccgggggcgcgccgggacccgaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgctttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcctgcaggcccgggggcgcgccctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccagggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctggctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 3 - DNA, 3510 bp, Artificial sequence
  Synthetic polynucleotide
atggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtag

SEQ ID NO: 4 - protein, 1169 aa, Artificial sequence
  Synthetic polypeptide
MEINNQNQCVPYNCLNNPESEILNVAIFSSEQVAEIHLKITRLILENFLPGGSFAFGLFDLIWGIFNEDQWSAFLRQVEELINQRITEFARGQAIQRLVGFGRSYDEYILALKEWENDPDNPASKERVRTRFRTTDDALLTGVPLMAIPGFELATLSVYAQSANLHLALLRDAVFFGERWGLTQTNINDLYSRLKNSIRDYTNHCVRFYNIGLGNLNVIRPEYYRFQRELTISVLDLVALFPNYDIRTYPIPTKSQLTREIYTDPIISPGAQAGYTLQDVLREPHLMDFLNRLIIYTGEYRGIRHWAGHEVESSRTGMMTNIRFPLYGTAATAEPTRFITPSTFPGLNLFYRTLSAPIFRDEPGANIIIRYRTSLVEGVGFIQPNNGEQLYRVRGTLDSLDQLPLEGESSLTEYSHRLCHVRFAQSLRNAEPLDYARVPMFSWTHRSATPTNTIDPDVITQIPLVKAHTLQSGTTVVKGPGFTGGDILRRTSGGPFAFSNVNLDWNLSQRYRARIRYASTTNLRMYVTIAGERIFAGQFNKTMNTGDPLTFQSFSYATIDTAFTFPTKASSLTVGADTFSSGNEVYVDRFELIPVTATFEAEYDLEKAQKAVNALFTSSNQIGLKTDVTDYHIDKVSNLVECLSDEFCLDEKRELSEKVKHAKRLCDERNLLQDPNFRGINRQPDRGWRGSTDITIQGGDDVFKENYVTLPGTFDECYPTYLYQKIDESKLKAYTRYELRGYIEDSQDLEIYLIRYNAKHETVNVPGTGSLWPLSAQSPIGKCGEPNRCATHLEWNPDLDCSCRDGEKCAHHSHHFSLDIDVGCTDLNEDLGVWVIFKIKTQDGHARLGNLEFLEEKPLVGEALARVKRAEKKWRDKREKLELETNIVYKEAKKSVDALFVNSQYDRLQADTNIAIIHAADKRVHSIREAYLPELSVIPGVNAAIFEELEGRIFTAYSLYDARNVIKNGDFNNGLSCWNVKGHVDVEEQNNHRSVLVVPEWEAEVSQEVRVCPGRGYILRVTAYKEGYGEGCVTIHEIEDNTDELKFSNCVEEEIYPNNTVTCNDYTATQEEYEGTYTSRNRGYDGAYESNSSVPADYASAYEEKAYTDGRRDNTCESNRGYGDYTPLPAGYVTKELEYFPETDKVWIEIGETEGTFIVDSVELLLMEE

SEQ ID NO: 5 - DNA, 1176 bp, Artificial sequence
  Synthetic polynucleotide
atgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaa

SEQ ID NO: 6 - protein, 391 aa, Artificial sequence
  Synthetic polypeptide
MQKLINSVQNYAWGSKTALTELYGMENPSSQPMAELWMGAHPKSSSRVQNAAGDIVSLRDVIESDKSTLLGEAVAKRFGELPFLFKVLCAAQPLSIQVHPNKHNSEIGFAKENAAGIPMDAAERNYKDPNHKPELVFALTPFLAMNAFREFSEIVSLLQPVAGAHPAIAHFLQQPDAERLSELFASLLNMQGEEKSRALAILKSALDSQQGEPWQTIRLISEFYPEDSGLFSPLLLNVVKLNPGEAMFLFAETPHAYLQGVALEVMANSDNVLRAGLTPKYIDIPELVANVKFEAKPANQLLTQPVKQGAELDFPIPVDDFAFSLHDLSDKETTISQQSAAILFCVEGDATLWKGSQQLQLKPGESAFIAANESPVTVKGHGRLARVYNKL

SEQ ID NO: 7 - DNA, 1176 bp, Artificial sequence
  Synthetic polynucleotide
atgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattgtgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaa

SEQ ID NO: 8 - DNA, 10641 bp, Artificial sequence
  Synthetic polynucleotide
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgctttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattgtgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 9 - DNA, 10641 bp, Artificial sequence
  Synthetic polynucleotide
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgctggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgctttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 10 - DNA, 10640 bp, Artificial sequence
  Synthetic polynucleotide
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgctggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgctttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 11 - DNA, 10642 bp, Artificial sequence
  Synthetic polynucleotide
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgctggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgctttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacacccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 12 - DNA, 10642 bp, Artificial sequence
  Synthetic polynucleotide
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgctggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgctttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 13 - DNA, 10642 bp, Artificial sequence
  Synthetic polynucleotide
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgctggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgctttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcatttcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 14 - DNA, 10641 bp, Artificial sequence
  Synthetic polynucleotide
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgctggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgctttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcatttcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 15 - DNA, 10643 bp, Artificial sequence
  Synthetic polynucleotide
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgctggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgctttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacacccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcatttcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 16 - DNA, 10643 bp, Artificial sequence
  Synthetic polynucleotide
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgctggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgctttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcatttcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 17 - DNA, 10641 bp, Artificial sequence
  Synthetic polynucleotide
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgctggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgctttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacacccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 18 - DNA, 10641 bp, Artificial sequence
  Synthetic polynucleotide
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgctggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgctttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 19 - DNA, 10643 bp, Artificial sequence
  Synthetic polynucleotide
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgctggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgctttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacacccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 20 - DNA, 10642 bp, Artificial sequence
  Synthetic polynucleotide
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgctggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgctttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacacccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcatttcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 21 - DNA, 10642 bp, Artificial sequence
  Synthetic polynucleotide
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgctggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgctttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcatttcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 22 - DNA, 10644 bp, Artificial sequence
  Synthetic polynucleotide
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgctggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgctttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacacccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcatttcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 23 - DNA, 10643 bp, Artificial sequence
  Synthetic polynucleotide
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgctggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgctttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacacccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcatttcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 24 - DNA, 10644 bp, Artificial sequence
  Synthetic polynucleotide
  misc_feature (324)..(324): n is c or absent
  misc_feature (7271)..(7271): n is c or absent
  misc_feature (7388)..(7388): n is c or absent
  misc_feature (7991)..(7991): n is t or absent
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctnccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgctggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgctttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacanccccctccacaccctctttccccaacctcgtgttgttcggagcgcacacacacacaaccagatctcccccaaatccacccgtcggcacctccgcttcaaggtacgccgctcgtcctnccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcanttcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 25 - DNA, 10642 bp, Artificial sequence
  Synthetic polynucleotide
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgcttttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatatttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctccttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 26 - DNA, 10644 bp, Artificial sequence
  Synthetic polynucleotide
  misc_feature (6015)..(6015): n is t or absent
  misc_feature (6471)..(6471): n is t or absent
  misc_feature (6684)..(6684): n is t or absent
  misc_feature (7388)..(7388): n is c or absent
  misc_feature (8399)..(8399): n is a or absent
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgcntttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatantttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctccnttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctnccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcnaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 27 - DNA, 10640 bp, Artificial sequence
  Synthetic polynucleotide
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaaccttttttttgttctatatatccatatcctagtactcagtagctctatgttttctggagactagtggcttgcttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttgggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagttttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 28 - DNA, 10643 bp, Artificial sequence
  Synthetic polynucleotide
  misc_feature (5944)..(5944): n is t or absent
  misc_feature (6015)..(6015): n is t or absent
  misc_feature (7387)..(7387): n is c or absent
  misc_feature (7577)..(7577): n is g or absent
  misc_feature (10056)..(10056): n is t or absent
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaaccnttttttttgttctatatatccatatcctagtactcagtagctctatgttttctggagactagtggcttgcnttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctnccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttngggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagntttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 29 - DNA, 10641 bp, Artificial sequence; identical to SEQ ID NO: 8
  Synthetic polynucleotide

SEQ ID NO: 30 - DNA, 10639 bp, Artificial sequence
  Synthetic polynucleotide
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtattttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgctttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgctttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattgtgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagcggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttaccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 31 - DNA, 10643 bp, Artificial sequence
  Synthetic polynucleotide
  misc_feature (347)..(347): n is c or absent
  misc_feature (1579)..(1579): n is t or absent
  misc_feature (7388)..(7388): n is c or absent
  misc_feature (7722)..(7722): n is t or absent
  misc_feature (8872)..(8872): n is c or absent
  misc_feature (9066)..(9066): n is c or absent
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatnccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtantttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgctttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctnccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcntttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattgtgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagncggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttanccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 32 - DNA, 18 bp, Artificial sequence
  Synthetic polynucleotide
ctccctcctcccccgtta

SEQ ID NO: 33 - DNA, 17 bp, Artificial sequence
  Synthetic polynucleotide
ctccctcctccccgtta

SEQ ID NO: 34 - DNA, 17 bp, Artificial sequence
  Synthetic polynucleotide
tgattctgcgggttggc

SEQ ID NO: 35 - DNA, 17 bp, Artificial sequence
  Synthetic polynucleotide
tgattctgctggttggc

SEQ ID NO: 36 - DNA, 31 bp, Artificial sequence
  Synthetic polynucleotide
taataaatagacaccccctccacaccctctt

SEQ ID NO: 37 - DNA, 32 bp, Artificial sequence
  Synthetic polynucleotide
taataaatagacacccccctccacaccctctt

SEQ ID NO: 38 - DNA, 23 bp, Artificial sequence
  Synthetic polynucleotide
ctcgtcctccccccccccccctc

SEQ ID NO: 39 - DNA, 24 bp, Artificial sequence
  Synthetic polynucleotide
ctcgtcctcccccccccccccctc

SEQ ID NO: 40 - DNA, 19 bp, Artificial sequence
  Synthetic polynucleotide
cggtcgttcattcgttcta

SEQ ID NO: 41 - DNA, 20 bp, Artificial sequence
  Synthetic polynucleotide
cggtcgttcatttcgttcta

SEQ ID NO: 42 - DNA, 29 bp, Artificial sequence
  Synthetic polynucleotide
agactagtggcttgctttttcgtatgtct

SEQ ID NO: 43 - DNA, 30 bp, Artificial sequence
  Synthetic polynucleotide
agactagtggcttgcttttttcgtatgtct

SEQ ID NO: 44 - DNA, 30 bp, Artificial sequence
  Synthetic polynucleotide
aaaaaattaccacatattttttttgtcaca

SEQ ID NO: 45 - DNA, 29 bp, Artificial sequence
  Synthetic polynucleotide
aaaaaattaccacatatttttttgtcaca

SEQ ID NO: 46 - DNA, 33 bp, Artificial sequence
  Synthetic polynucleotide
tagtgtgcatgtgttctcctttttttttgcaaa

SEQ ID NO: 47 - DNA, 32 bp, Artificial sequence
  Synthetic polynucleotide
tagtgtgcatgtgttctccttttttttgcaaa

SEQ ID NO: 48 - DNA, 32 bp, Artificial sequence
  Synthetic polynucleotide
gtacgccgctcgtcctccccccccccccctct

SEQ ID NO: 49 - DNA, 33 bp, Artificial sequence
  Synthetic polynucleotide
gtacgccgctcgtcctcccccccccccccctct

SEQ ID NO: 50 - DNA, 34 bp, Artificial sequence
  Synthetic polynucleotide
gatctccgatcatgcaaaaactcattaactcagt

SEQ ID NO: 51 - DNA, 35 bp, Artificial sequence
  Synthetic polynucleotide
gatctccgatcatgcaaaaaactcattaactcagt

SEQ ID NO: 52 - DNA, 23 bp, Artificial sequence
  Synthetic polynucleotide
cttatgcagaacctttttttttg

SEQ ID NO: 53 - DNA, 22 bp, Artificial sequence
  Synthetic polynucleotide
cttatgcagaaccttttttttg

SEQ ID NO: 54 - DNA, 31 bp, Artificial sequence
  Synthetic polynucleotide
ggagactagtggcttgctttttcgtatgtct

SEQ ID NO: 55 - DNA, 30 bp, Artificial sequence
  Synthetic polynucleotide
ggagactagtggcttgcttttcgtatgtct

SEQ ID NO: 56 - DNA, 30 bp, Artificial sequence
  Synthetic polynucleotide
acgccgctcgtcctccccccccccccctct

SEQ ID NO: 57 - DNA, 31 bp, Artificial sequence
  Synthetic polynucleotide
acgccgctcgtcctcccccccccccccctct

SEQ ID NO: 58 - DNA, 31 bp, Artificial sequence
  Synthetic polynucleotide
gccagtgtttctctttggggaatcctgggat

SEQ ID NO: 59 - DNA, 30 bp, Artificial sequence
  Synthetic polynucleotide
gccagtgtttctctttgggaatcctgggat

SEQ ID NO: 60 - DNA, 30 bp, Artificial sequence
  Synthetic polynucleotide
actaacaattagtttcagtgcattcaaaca

SEQ ID NO: 61 - DNA, 31 bp, Artificial sequence
  Synthetic polynucleotide
actaacaattagttttcagtgcattcaaaca

SEQ ID NO: 62 - DNA, 33 bp, Artificial sequence
  Synthetic polynucleotide
ttataaattggcttcatcccctccttgcctcat

SEQ ID NO: 63 - DNA, 32 bp, Artificial sequence
  Synthetic polynucleotide
ttataaattggcttcatccctccttgcctcat

SEQ ID NO: 64 - DNA, 24 bp, Artificial sequence
  Synthetic polynucleotide
catatatcatgtatttttttttgg

SEQ ID NO: 65 - DNA, 25 bp, Artificial sequence
  Synthetic polynucleotide
catatatcatgtattttttttttgg

SEQ ID NO: 66 - DNA, 29 bp, Artificial sequence
  Synthetic polynucleotide
tacgccgctcgtcctccccccccccccct

SEQ ID NO: 67 - DNA, 30 bp, Artificial sequence
  Synthetic polynucleotide
tacgccgctcgtcctcccccccccccccct

SEQ ID NO: 68 - DNA, 36 bp, Artificial sequence
  Synthetic polynucleotide
catcttttcatgcttttttttgtcttggttgtgatg

SEQ ID NO: 69 - DNA, 35 bp, Artificial sequence
  Synthetic polynucleotide
catcttttcatgctttttttgtcttggttgtgatg

SEQ ID NO: 70 - DNA, 31 bp, Artificial sequence
  Synthetic polynucleotide
cctgttcaaagtattatgcgcagcacagcca

SEQ ID NO: 71 - DNA, 31 bp, Artificial sequence
  Synthetic polynucleotide
cctgttcaaagtattgtgcgcagcacagcca

SEQ ID NO: 72 - DNA, 31 bp, Artificial sequence
  Synthetic polynucleotide
gtctccctactccagccggtcgcaggtgcac

SEQ ID NO: 73 - DNA, 30 bp, Artificial sequence
  Synthetic polynucleotide
gtctccctactccagcggtcgcaggtgcac

SEQ ID NO: 74 - DNA, 30 bp, Artificial sequence
  Synthetic polynucleotide
aatttctgaattttacccggaagacagcgg

SEQ ID NO: 75 - DNA, 29 bp, Artificial sequence
  Synthetic polynucleotide
aatttctgaattttaccggaagacagcgg

(19) United States
(12) Patent Application Publication
(10) Pub. No.: US 2024/0240199 A1
(43) Pub. Date: Jul. 18, 2024

(54) NUCLEIC ACID MOLECULES FOR CONFERRING INSECTICIDAL PROPERTIES IN PLANTS

(71) Applicant: SYNGENTA CROP PROTECTION AG, Basel (CH)

(72) Inventors: Ailing Zhou, Research Triangle Park, NC (US); Kasimalai Azhakanandam, Research Triangle Park, NC (US); Jared Conville, Research Triangle Park, NC (US); Yan Zhang, Research Triangle Park, NC (US)

(73) Assignee: Syngenta Crop Protection AG, Basel (CH)

(21) Appl. No.: 18/289,473

(22) PCT Filed: May 3, 2022
(86) PCT No.: PCT/US22/27372

§ 371 Date: Nov. 3, 2023

Related U.S. Application Data
(60) Provisional application No. 63/183,672, filed on May 4, 2021.

(51) Int. Cl. C12N 15/82; C07K 14/325; C12Q 1/6895

(52) U.S. Cl. CPC C12N 15/8286; C07K 14/325; C12Q 1/6895; C12Q 2600/13

(57) ABSTRACT

The present disclosure is related to a nucleic acid sequence which confers expression of insecticidal proteins when introduced into a cell, as well as related compositions and methods of use thereof. In some aspects, the disclosure provides a plant comprising the nucleic acid sequence.

### CROSS-REFERENCE TO RELATED APPLICATIONS

This application claims priority to U.S. Provisional Application No. 63/183,672, filed on May 4, 2021, the entire contents of which are incorporated by reference herein.

### FIELD OF THE INVENTION

The present invention generally relates to nucleic acid sequences which confer expression of insecticidal proteins when introduced into a cell or plant, as well as related compositions and methods.

### SEQUENCE LISTING

This application is accompanied by a sequence listing in ASCII text format entitled “82347-PCT_ST25.txt,” created Apr. 14, 2022, which is approximately 395 kilobytes in size. This sequence listing is incorporated herein by reference in its entirety. This sequence listing is submitted herewith via EFS-Web, and is in compliance with 37 C.F.R. § 1.824(a)(2)-(6) and (b).

### BACKGROUND

Plant pests are a major factor in the loss of the world's important agricultural crops, including maize. Plant pests are mainly controlled by intensive applications of chemical pesticides. Good pest control can thus be reached, but these chemicals can sometimes also affect beneficial organisms. Another problem resulting from the wide use of chemical pesticides is the appearance of resistant insect varieties. This has been partially alleviated by various resistance management practices, but there is an increasing need for alternative pest control strategies. One such alternative includes the expression of foreign genes encoding insecticidal proteins in transgenic plants. This approach has provided an efficient means of protection against selected insect pests, and transgenic plants expressing insecticidal toxins have been commercialized, allowing farmers to reduce applications of chemical insecticides.

Bacillus thuringiensis (Bt) Cry proteins (also called delta-endotoxins) are proteins that form a crystalline matrix in Bacillus that are known to possess insecticidal activity when ingested by certain insects. Genes coding for Cry proteins have been isolated and their expression in crop plants have been shown to provide another tool for the control of economically important insect pests.

Although the usage of transgenic plants expressing Cry proteins is another tool in the insect control toolbox, it is still susceptible to resistance breakdown. Insect pests that now have resistance against the Cry proteins expressed in certain transgenic plants are known. For example, fall armyworm (Spodoptera frugiperda) has documented field-evolved resistance to Cry1F, Cry1A.105 and Cry2Ab2 in certain countries. As a result, there is a need for additional insecticidal proteins to address the resistance issues.

Creating new insecticidal protein expression cassettes for use in transgenic plants is a challenging endeavor as the expression cassette must express enough protein(s) within the transgenic plant to have the desired activity (e.g., insecticidal activity) without causing negative effects on the plant itself (e.g., reduced yield, sterility, stunting, etc.).

Provided herein are nucleic acid sequences and related compositions and methods of use to address the aforementioned needs.

### SUMMARY

In some aspects, the disclosure provides a nucleic acid molecule that expresses one or more insecticidal proteins. As described herein, an expression cassette was created (SEQ ID NO: 1) which encodes an eCry1Gb.1Ig protein (SEQ ID NO: 4). This expression cassette, when transformed into plants, confers insecticidal activity against Lepidoptera species, e.g., Spodoptera frugiperda (fall armyworm).

Accordingly, in some aspects, the disclosure provides a nucleic acid molecule comprising a nucleic acid sequence that is at least 90% identical to SEQ ID NO: 1 (e.g., at least 90% identical to SEQ ID NO: 1, at least 91% identical to SEQ ID NO: 1, at least 92% identical to SEQ ID NO: 1, at least 93% identical to SEQ ID NO: 1, at least 94% identical to SEQ ID NO: 1, at least 95% identical to SEQ ID NO: 1, at least 96% identical to SEQ ID NO: 1, at least 97% identical to SEQ ID NO: 1, at least 98% identical to SEQ ID NO: 1, at least 99% identical to SEQ ID NO: 1, or at least 99.5% identical to SEQ ID NO: 1), or the complement thereof. In some embodiments, the nucleic acid molecule encodes the same protein(s) that are encoded by SEQ ID NO: 1. In some embodiments, the nucleic acid sequence comprises any one of SEQ ID NOs: 1 or 8 to 31 or any one or more of the variants in Table 3. In some embodiments, the nucleic acid molecule encodes protein(s) that is/are insecticidal against one or more Lepidopteran pests, e.g., insecticidal against at least Spodoptera frugiperda (fall armyworm). In some embodiments, the nucleic acid molecule encodes protein(s) that is/are insecticidal against at least two (e.g., 2, 3, or 4) of Spodoptera frugiperda (fall armyworm), Mythimna separata (oriental armyworm), Spodoptera litura (common cutworm/oriental leafworm), and Ostrinia furnacalis (Asian corn borer). In some embodiments, the nucleic acid molecule is isolated.

In some embodiments, the disclosure provides a nucleic acid molecule comprising a nucleic acid sequence that is at least 95% identical to SEQ ID NO: 1 (e.g., at least 95% identical to SEQ ID NO: 1, at least 96% identical to SEQ ID NO: 1, at least 97% identical to SEQ ID NO: 1, at least 98% identical to SEQ ID NO: 1, at least 99% identical to SEQ ID NO: 1, or at least 99.5% identical to SEQ ID NO: 1), or the complement thereof, wherein the nucleic acid sequence encodes a polypeptide comprising the sequence of SEQ ID NO: 4 or encodes polypeptides comprising the sequences of SEQ ID NO: 4 and 6. In some embodiments, the nucleic acid sequence comprises SEQ ID NO: 3 or SEQ ID NO: 3 and 5 or a variant thereof of any of the foregoing comprising one or more silent mutations. In some embodiments, the nucleic acid sequence comprises any one of SEQ ID NOs: 1 or 8 to 31 or any one or more of the variants in Table 3 or a variant thereof of any of the foregoing comprising one or more silent mutations or other mutations that do not substantially affect the function of SEQ ID NO: 1.

In some aspects, the disclosure provides a recombinant nucleic acid vector comprising the nucleic acid molecule of any of the above-mentioned embodiments or any other embodiment described herein (e.g., comprising any one of SEQ ID NOs: 1 or 8 to 31 or any one or more of the variants in Table 3). In some embodiments, the vector is a binary vector. In some embodiments, the vector is a plasmid. In some embodiments, the vector is present in a host cell.

In some aspects, the disclosure provides a transgenic host cell comprising the nucleic acid molecule of any of the above-mentioned embodiments or any other embodiment described herein (e.g., comprising any one of SEQ ID NOs: 1 or 8 to 31 or any one or more of the variants in Table 3). In some embodiments, the cell is a plant cell, a yeast cell, a bacterial cell or an insect cell. In some embodiments, the cell is a bacterial cell or a plant cell. In some embodiments, the cell is a bacterial cell and the bacterial cell is an Escherichia coli. Bacillus thuringiensis. Bacillus subtilis. Bacillus megaterium. Bacillus cereus. Agrobacterium ssp. or a Pseudomonas ssp. cell. In some embodiments, the cell is a plant cell and the plant cell is a maize, sorghum, wheat, sunflower, tomato, crucifers, oat, turf grass, pasture grass, peppers, potato, cotton, rice, soybean, sugarcane, sugar beet, tobacco, barley, or oilseed rape cell. In some embodiments, the plant cell is a maize cell. In some embodiments, the plant cell is present in a plant. In some embodiments, the plant cell is isolated. In some embodiments, the plant cell is capable of regenerating a plant. In some embodiments, the plant cell is incapable of regenerating a whole plant.

In some aspects, the disclosure provides a transgenic plant comprising the nucleic acid molecule of any of the above-mentioned embodiments or any other embodiment described herein (e.g., comprising any one of SEQ ID NOs: 1 or 8 to 31 or any one or more of the variants in Table 3). In some embodiments, the plant is a monocot plant. In some embodiments, the plant is a dicot plant. In some embodiments, the plant is selected from the group consisting of maize, sorghum, wheat, sunflower, tomato, crucifers, oat, turf grass, pasture grass, peppers, potato, cotton, rice, soybean, sugarcane, sugar beet, tobacco, barley, and oilseed rape. In some embodiments, the plant is a maize plant. In some embodiments, the plant is a whole plant. In some embodiments, the plant is a transgenic whole maize plant comprising a nucleic acid molecule comprising any one of SEQ ID NOs: 1 or 8 to 31 or any one or more of the variants in Table 3. In some embodiments, the plant is insecticidal against at least Spodoptera frugiperda (fall armyworm). In some embodiments, the plant is insecticidal against at least two (e.g., 2, 3, or 4) of Spodoptera frugiperda (fall armyworm), Mythimna separata (oriental armyworm), Spodoptera litura (common cutworm/oriental leafworm), and Ostrinia furnacalis (Asian corn borer). In some embodiments, the plant has enhanced insecticidal properties, e.g., against at least Spodoptera frugiperda (fall armyworm), relative to a control plant, e.g., that does not comprise the nucleic acid molecule. In some aspects, the disclosure provides a progeny of any generation of the plant, wherein the progeny comprises the nucleic acid molecule. In some aspects, the disclosure provides a propagule of the plant, wherein the propagule comprises the nucleic acid molecule. In some aspects, the disclosure provides a plant part of the plant, wherein the plant part comprises the nucleic acid molecule. In some embodiments, the plant part is an embryo, pollen, ovule, seed, leaf, flower, branch, fruit, kernel, ear, cob, husk, stalk, root, root tip, anther, tuber, or rhizome. In some embodiments, the plant part is a seed.

In some aspects, the disclosure provides a method of producing a transgenic plant with enhanced insecticidal properties, comprising introducing the nucleic acid molecule of any of the above-mentioned embodiments or any other embodiment described herein (e.g., comprising any one of SEQ ID NOs: 1 or 8 to 31 or any one or more of the variants in Table 3) into a plant thereby producing a transgenic plant, wherein the nucleic acid molecule expresses effective insect-controlling amounts of protein. In some embodiments, the effective insect-controlling amounts of protein are effective for controlling at least Spodoptera frugiperda (fall armyworm). In some embodiments, the effective insect-controlling amounts of protein are effective for controlling at least two (e.g., 2, 3, or 4) of Spodoptera frugiperda (fall armyworm), Mythimna separata (oriental armyworm), Spodoptera litura (common cutworm/oriental leafworm), and Ostrinia furnacalis (Asian corn borer).

In some aspects, the disclosure provides a method of producing a transgenic plant with enhanced insecticidal properties, comprising the steps of: (a) providing the nucleic acid molecule of any of the above-mentioned embodiments or any other embodiment described herein (e.g., comprising any one of SEQ ID NOs: 1 or 8 to 31 or any one or more of the variants in Table 3); (b) introducing into a plant, tissue culture, or a plant cell the nucleic acid molecule of step (a) to obtain a transformed plant, transformed tissue culture, or a transformed cell having enhanced insecticidal properties; and (c) growing the transformed plant or regenerating a transformed plant from the transformed tissue culture or transformed plant cell, so a transgenic plant with enhanced insecticidal properties is produced. In some embodiments, the enhanced insecticidal properties are enhanced insecticidal properties against at least Spodoptera frugiperda (fall armyworm). In some embodiments, the enhanced insecticidal properties are enhanced insecticidal properties against at least two (e.g., 2, 3, or 4) of Spodoptera frugiperda (fall armyworm), Mythimna separata (oriental armyworm), Spodoptera litura (common cutworm/oriental leafworm), and Ostrinia furnacalis (Asian corn borer). In some embodiments, the transgenic plant is a transgenic maize plant.

In some aspects, the disclosure provides a method of producing transgenic seed, comprising the steps of: (a) obtaining a fertile transgenic plant of any of the above-mentioned embodiments or any other embodiment described herein (e.g., comprising any one of SEQ ID NOs: 1 or 8 to 31 or any one or more of the variants in Table 3); and (b) growing the plant under appropriate conditions to produce the transgenic seed. In some embodiments, the transgenic seed is transgenic maize seed.

In some aspects, the disclosure provides a method of producing progeny of any generation of a fertile transgenic plant with enhanced insecticidal properties, comprising the steps of: (a) obtaining a fertile transgenic plant with enhanced insecticidal properties comprising the nucleic acid molecule of any of the above-mentioned embodiments or any other embodiment described herein (e.g., comprising any one of SEQ ID NOs: 1 or 8 to 31 or any one or more of the variants in Table 3); (b) collecting transgenic seed from the transgenic plant; (c) planting the collected transgenic seed; and (d) growing the progeny transgenic plants from the seed, wherein the progeny has enhanced insecticidal properties relative to a non-transformed plant. In some embodiments, the progeny plant are maize plants.

In some aspects, the disclosure provides a method for producing a transgenic plant with enhanced insecticidal properties, comprising the steps of sexually crossing a first parent plant with a second parent plant, wherein the first or second parent plant is the plant of any of the above-mentioned embodiments or any other embodiment described herein (e.g., comprising any one of SEQ ID NOs: 1 or 8 to 31 or any one or more of the variants in Table 3), to produce a first generation progeny plant that comprises the nucleic acid molecule. In some embodiments, the enhanced insecticidal properties are enhanced insecticidal properties against at least Spodoptera frugiperda (fall armyworm). In some embodiments, the enhanced insecticidal properties are enhanced insecticidal properties against at least two (e.g., 2, 3, or 4) of Spodoptera frugiperda (fall armyworm), Mythimna separata (oriental armyworm), Spodoptera litura (common cutworm/oriental leafworm), and Ostrinia furnacalis (Asian corn borer). In some embodiments, the first generation progeny plant is a maize plant.

In some aspects, the disclosure provides a method for producing a transgenic plant with enhanced insecticidal properties, comprising the steps of: (a) sexually crossing a first parent plant with a second parent plant, wherein the first or second parent plant is the plant of any of the above-mentioned embodiments or any other embodiment described herein (e.g., comprising any one of SEQ ID NOs: 1 or 8 to 31 or any one or more of the variants in Table 3); and (b) selecting a first generation progeny plant with enhanced insecticidal properties, wherein the selected progeny plant comprises the nucleic acid molecule. In some embodiments, the enhanced insecticidal properties are enhanced insecticidal properties against at least Spodoptera frugiperda (fall armyworm). In some embodiments, the enhanced insecticidal properties are enhanced insecticidal properties against at least two (e.g., 2, 3, or 4) of Spodoptera frugiperda (fall armyworm), Mythimna separata (oriental armyworm), Spodoptera litura (common cutworm/oriental leafworm), and Ostrinia furnacalis (Asian corn borer). In some embodiments, the first generation progeny plant is a maize plant. In some embodiments, the method further comprises the steps of: (a) selfing the first generation progeny plant, thereby producing a plurality of second generation progeny plants; and (b) selecting from the second generation progeny plants a plant with enhanced insecticidal properties, wherein the selected second generation progeny plants comprise the nucleic acid molecule.

In some aspects, the disclosure provides a method of controlling a lepidopteran pest comprising feeding the pest a plant or plant part comprising the nucleic acid molecule of any of the above-mentioned embodiments or any other embodiment described herein (e.g., comprising any one of SEQ ID NOs: 1 or 8 to 31 or any one or more of the variants in Table 3). In some embodiments, the lepidopteran pest is Spodoptera frugiperda (fall armyworm). In some embodiments, the lepidopteran pest is at least two (e.g., 2, 3, or 4) of Spodoptera frugiperda (fall armyworm), Mythimna separata (oriental armyworm), Spodoptera litura (common cutworm/oriental leafworm), and Ostrinia furnacalis (Asian corn borer). In some embodiments, the plant or plant part is a maize plant or maize plant part.

In some aspects, the disclosure provides a method of producing a commodity plant product, the method comprising using the plant of any of the above-mentioned embodiments or any other embodiment described herein (e.g., comprising any one of SEQ ID NOs: 1 or 8 to 31 or any one or more of the variants in Table 3) to produce said commodity plant product therefrom. In some embodiments, the plant is a maize plant. In some embodiments, the commodity plant product is a grain, starch, seed oil, syrup, flour, meal, starch, cereal, or protein.

In some aspects, the disclosure provides a method of detecting the presence of a nucleic acid molecule in a sample, the method comprising: (a) contacting the sample with a pair of primers that, when used in a nucleic-acid amplification reaction with DNA comprising the nucleic acid molecule of any of the above-mentioned embodiments or any other embodiment described herein (e.g., comprising SEQ ID NO: any one of SEQ ID NOs: 1 or 8 to 31 or any one or more of the variants in Table 3), produces an amplicon that is diagnostic for the nucleic acid molecule; (b) performing a nucleic acid amplification reaction, thereby producing the amplicon; and (c) detecting the amplicon. In some embodiments, the pair of primers is a first primer and a second primer wherein the first primer comprises at least 10 contiguous nucleotides that are complementary to any one of SEQ ID NOs: 1 or 8 to 31 or any one or more of the variants in Table 3 and the second primer comprises at least 10 contiguous nucleotides that are complementary to the reverse complement of any one of SEQ ID NOs: 1 or 8 to 31 or any one or more of the variants in Table 3. In some embodiments, the first and second primer are between 10-30 nucleotides in length. In some embodiments, the sample is a sample obtained from a maize plant part or cell.

In some aspects, the disclosure provides a method of detecting the presence of a nucleic acid molecule in a sample, the method comprising: (a) contacting the sample with a probe that hybridizes under high stringency conditions with DNA comprising the nucleic acid molecule of any of the above-mentioned embodiments or any other embodiment described herein (e.g., comprising any one of SEQ ID NOs: 1 or 8 to 31 or any one or more of the variants in Table 3) and does not hybridize under high stringency conditions with DNA of a control maize plant not comprising the nucleic acid molecule; (b) subjecting the sample and probe to high stringency hybridization conditions; and (c) detecting hybridization of the probe to the nucleic acid molecule. In some embodiments, the probe comprises at least 10 contiguous nucleotides that are complementary to any one of SEQ ID NOs: 1 or 8 to 31 or any one or more of the variants in Table 3 or the reverse complement thereof. In some embodiments, the probe is between 10-50 nucleotides in length. In some embodiments, the sample is a sample obtained from a maize plant part or cell.

In some aspects, the disclosure provides a pair of polynucleotide primers comprising a first polynucleotide primer and a second polynucleotide primer which function together in the presence of the nucleic acid molecule of any of the above-mentioned embodiments or any other embodiment described herein (e.g., comprising any one of SEQ ID NOs: 1 or 8 to 31 or any one or more of the variants in Table 3) in a sample to produce an amplicon diagnostic for the presence of the nucleic acid molecule in a sample. In some embodiments, the sample is a sample obtained from a maize plant part or cell. In some embodiments, the first polynucleotide primer comprises at least 10 contiguous nucleotides that are complementary to any one of SEQ ID NOs: 1 or 8 to 31 or any one or more of the variants in Table 3 and the second polynucleotide primer comprises at least 10 contiguous nucleotides that are complementary to the reverse complement of any one of SEQ ID NOs: 1 or 8 to 31 or any one or more of the variants in Table 3. In some embodiments, the first and second primer are between 10-30 nucleotides in length.

In some aspects, the disclosure provides a kit for detecting the nucleic acid molecule of any of the above-mentioned embodiments or any other embodiment described herein (e.g., comprising any one of SEQ ID NOs: 1 or 8 to 31 or any one or more of the variants in Table 3), the kit comprising at least one nucleic acid molecule of sufficient length of contiguous nucleotides to function as a primer or probe in a nucleic acid detection method, and which upon amplification of or hybridization to a target nucleic acid sequence in a sample followed by detection of the amplicon or hybridization to the target sequence, are diagnostic for the presence of the nucleic acid molecule. In some embodiments, the at least one nucleic acid molecule comprises at least 10 contiguous nucleotides that are complementary to any one of SEQ ID NOs: 1 or 8 to 31 or any one or more of the variants in Table 3. In some embodiments, the at least one nucleic acid molecule comprises a pair of primers, wherein the first polynucleotide primer comprises at least 10 contiguous nucleotides that are complementary to any one of SEQ ID NOs: 1 or 8 to 31 or any one or more of the variants in Table 3 and the second polynucleotide primer comprises at least 10 contiguous nucleotides that are complementary to the reverse complement of any one of SEQ ID NOs: 1 or 8 to 31 or any one or more of the variants in Table 3. In some embodiments, the first and second primer are between 10-30 nucleotides in length. In some embodiments, the at least one nucleic acid molecule comprises a probe that comprises at least 10 contiguous nucleotides that are complementary to any one of SEQ ID NOs: 1 or 8 to 31 or any one or more of the variants in Table 3 or the reverse complement thereof. In some embodiments, the probe is between 10-50 nucleotides in length.

In some aspects, the disclosure provides a method, comprising introducing a modification into a nucleic acid molecule, transgenic host cell, or transgenic plant of any one of the above-mentioned embodiments, thereby producing a modified nucleic acid molecule, transgenic host cell, or a modified transgenic plant. In some embodiments, the modification is a deletion, an insertion, a substitution, a duplication, or inversion or a combination thereof. In some embodiments, the modification comprises deletion of a portion or all of a selectable marker coding sequence present in the nucleic acid molecule (e.g., PMI). In some embodiments, the modification is introduced using a nuclease or homologous recombination, or a combination thereof. In some embodiments, the nuclease is a CRISPR-Cas nuclease. In some embodiments, the method further comprises producing a plant from the modified transgenic host cell and selfing or crossing the plant with another plant, thereby producing a modified transgenic progeny plant. In some embodiments, the method further comprises selfing or crossing the modified transgenic plant with another plant, thereby producing a modified transgenic progeny plant. In some embodiments, the method further comprises selfing or outcrossing the modified transgenic progeny plant for at least one additional generation.

### BRIEF DESCRIPTION OF THE DRAWINGS

FIG. 1 is a diagram of binary vector 24795, whose nucleic acid sequence is SEQ ID NO:2.

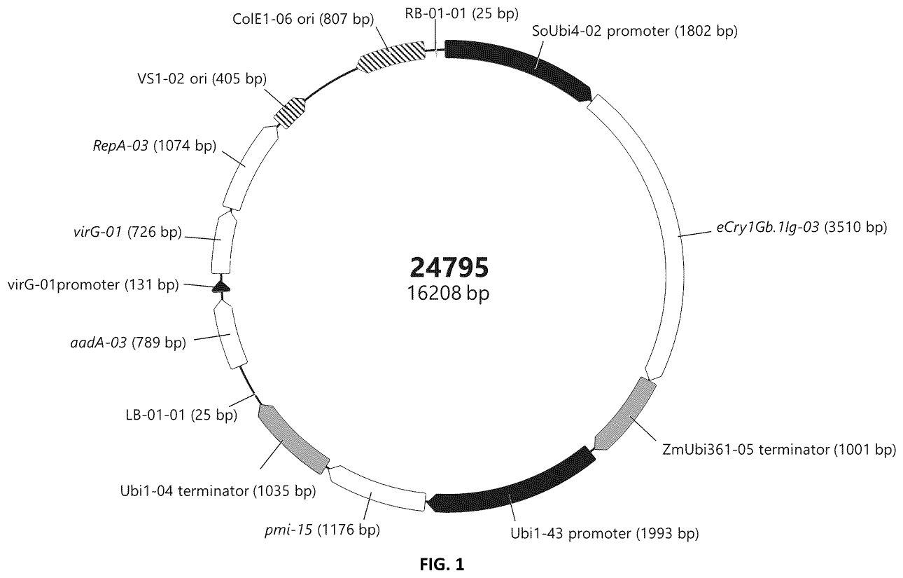

### BRIEF DESCRIPTION OF THE SEQUENCES IN THE SEQUENCE LISTING

SEQ ID NO: 1 is the nucleic acid sequence of the expression cassette encoding an eCry1Gb.1Ig protein (SEQ ID NO: 4) as well as PMI (SEQ ID NO: 6) as a selectable marker.

SEQ ID NO: 2 is the nucleic acid sequence of the binary vector 24795, which includes the expression cassette of SEQ ID NO: 1.

SEQ ID NO: 3 is the nucleic acid sequence of a coding sequence encoding eCry1Gb.1Ig.

SEQ ID NO: 4 is the amino acid sequence of eCry1Gb.1Ig.

SEQ ID NO: 5 is the nucleic acid sequence of a coding sequence encoding PMI.

SEQ ID NO: 6 is the amino acid sequence of PMI.

SEQ ID NO: 7 is the nucleic acid sequence of a coding sequence encoding PMI which has a silent mutation at one nucleotide position relative to SEQ ID NO: 5.

SEQ ID NO: 8 is the nucleic acid sequence of the expression cassette encoding an eCry1Gb.1Ig protein (SEQ ID NO: 4) as well as PMI (SEQ ID NO: 6) as a selectable marker and containing the silent mutation in SEQ ID NO: 7.

SEQ ID NO: 9 is the nucleic acid sequence of the expression cassette encoding an eCry1Gb.1Ig protein (SEQ ID NO: 4) as well as PMI (SEQ ID NO: 6) as a selectable marker and containing an additional mutation relative to SEQ ID NO: 1.

SEQ ID NO: 10 is the nucleic acid sequence of the expression cassette encoding an eCry1Gb.1Ig protein (SEQ ID NO: 4) as well as PMI (SEQ ID NO: 6) as a selectable marker and containing additional mutations relative to SEQ ID NO: 1.

SEQ ID NO: 11 is the nucleic acid sequence of the expression cassette encoding an eCry1Gb.1Ig protein (SEQ ID NO: 4) as well as PMI (SEQ ID NO: 6) as a selectable marker and containing additional mutations relative to SEQ ID NO: 1.

SEQ ID NO: 12 is the nucleic acid sequence of the expression cassette encoding an eCry1Gb.1Ig protein (SEQ ID NO: 4) as well as PMI (SEQ ID NO: 6) as a selectable marker and containing additional mutations relative to SEQ ID NO: 1.

SEQ ID NO: 13 is the nucleic acid sequence of the expression cassette encoding an eCry1Gb.1Ig protein (SEQ ID NO: 4) as well as PMI (SEQ ID NO: 6) as a selectable marker and containing additional mutations relative to SEQ ID NO: 1.

SEQ ID NO: 14 is the nucleic acid sequence of the expression cassette encoding an eCry1Gb.1Ig protein (SEQ ID NO: 4) as well as PMI (SEQ ID NO: 6) as a selectable marker and containing additional mutations relative to SEQ ID NO: 1.

SEQ ID NO: 15 is the nucleic acid sequence of the expression cassette encoding an eCry1Gb.1Ig protein (SEQ ID NO: 4) as well as PMI (SEQ ID NO: 6) as a selectable marker and containing additional mutations relative to SEQ ID NO: 1.

SEQ ID NO: 16 is the nucleic acid sequence of the expression cassette encoding an eCry1Gb.1Ig protein (SEQ ID NO: 4) as well as PMI (SEQ ID NO: 6) as a selectable marker and containing additional mutations relative to SEQ ID NO: 1.

SEQ ID NO: 17 is the nucleic acid sequence of the expression cassette encoding an eCry1Gb.1Ig protein (SEQ ID NO: 4) as well as PMI (SEQ ID NO: 6) as a selectable marker and containing additional mutations relative to SEQ ID NO: 1.

SEQ ID NO: 18 is the nucleic acid sequence of the expression cassette encoding an eCry1Gb.1Ig protein (SEQ ID NO: 4) as well as PMI (SEQ ID NO: 6) as a selectable marker and containing additional mutations relative to SEQ ID NO: 1.

SEQ ID NO: 19 is the nucleic acid sequence of the expression cassette encoding an eCry1Gb.1Ig protein (SEQ ID NO: 4) as well as PMI (SEQ ID NO: 6) as a selectable marker and containing additional mutations relative to SEQ ID NO: 1.

SEQ ID NO: 20 is the nucleic acid sequence of the expression cassette encoding an eCry1Gb.1Ig protein (SEQ ID NO: 4) as well as PMI (SEQ ID NO: 6) as a selectable marker and containing additional mutations relative to SEQ ID NO: 1.

SEQ ID NO: 21 is the nucleic acid sequence of the expression cassette encoding an eCry1Gb.1Ig protein (SEQ ID NO: 4) as well as PMI (SEQ ID NO: 6) as a selectable marker and containing additional mutations relative to SEQ ID NO: 1.

SEQ ID NO: 22 is the nucleic acid sequence of the expression cassette encoding an eCry1Gb.1Ig protein (SEQ ID NO: 4) as well as PMI (SEQ ID NO: 6) as a selectable marker and containing additional mutations relative to SEQ ID NO: 1.

SEQ ID NO: 23 is the nucleic acid sequence of the expression cassette encoding an eCry1Gb.1Ig protein (SEQ ID NO: 4) as well as PMI (SEQ ID NO: 6) as a selectable marker and containing additional mutations relative to SEQ ID NO: 1.

SEQ ID NO: 24 is the nucleic acid sequence of the expression cassette encoding an eCry1Gb.1Ig protein (SEQ ID NO: 4) as well as PMI (SEQ ID NO: 6) as a selectable marker and containing additional mutations relative to SEQ ID NO: 1.

SEQ ID NO: 25 is the nucleic acid sequence of the expression cassette encoding an eCry1Gb.1Ig protein (SEQ ID NO: 4) as well as PMI (SEQ ID NO: 6) as a selectable marker and containing additional mutations relative to SEQ ID NO: 1.

SEQ ID NO: 26 is the nucleic acid sequence of the expression cassette encoding an eCry1Gb.1Ig protein (SEQ ID NO: 4) as well as PMI (SEQ ID NO: 6) as a selectable marker and containing additional mutations relative to SEQ ID NO: 1.

SEQ ID NO: 27 is the nucleic acid sequence of the expression cassette encoding an eCry1Gb.1Ig protein (SEQ ID NO: 4) as well as PMI (SEQ ID NO: 6) as a selectable marker and containing additional mutations relative to SEQ ID NO: 1.

SEQ ID NO: 28 is the nucleic acid sequence of the expression cassette encoding an eCry1Gb.1Ig protein (SEQ ID NO: 4) as well as PMI (SEQ ID NO: 6) as a selectable marker and containing additional mutations relative to SEQ ID NO: 1.

SEQ ID NO: 29 is the nucleic acid sequence of the expression cassette encoding an eCry1Gb.1Ig protein (SEQ ID NO: 4) as well as PMI (SEQ ID NO: 6) as a selectable marker and containing an additional mutation relative to SEQ ID NO: 1.

SEQ ID NO: 30 is the nucleic acid sequence of the expression cassette encoding an eCry1Gb.1Ig protein (SEQ ID NO: 4) as well as PMI (SEQ ID NO: 6) as a selectable marker and containing additional mutations relative to SEQ ID NO: 1.

SEQ ID NO: 31 is the nucleic acid sequence of the expression cassette encoding an eCry1Gb.1Ig protein (SEQ ID NO: 4) as well as PMI (SEQ ID NO: 6) as a selectable marker and containing additional mutations relative to SEQ ID NO: 1.

SEQ ID NOs: 32-75 are in Table 3.

### DETAILED DESCRIPTION

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the disclosure contemplates that in some embodiments, any feature or combination of features set forth herein can be excluded or omitted. In addition, numerous variations and additions to the various embodiments suggested herein will be apparent to those skilled in the art in light of the instant disclosure, which do not depart from the instant disclosure. Hence, the following descriptions are intended to illustrate some particular embodiments of the disclosure, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Nucleotide sequences provided herein are presented in the 5′ to 3′ direction, from left to right and are presented using the standard code for representing nucleotide bases as set forth in 37 CFR §§ 1.821-1.825 and the World Intellectual Property Organization (WIPO) Standard ST.25, for example: adenine (A), cytosine (C), thymine (T), and guanine (G).

Amino acids are likewise indicated using the WIPO Standard ST.25, for example: alanine (Ala; A), arginine (Arg; R), asparagine (Asn; N), aspartic acid (Asp; D), cysteine (Cys; C), glutamine (Gln; Q), glutamic acid (Glu; E), glycine (Gly; G), histidine (His; H), isoleucine (Ile; 1), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Unless the context indicates otherwise, it is specifically intended that the various features of the disclosure described herein can be used in any combination. Moreover, the present disclosure also contemplates that in some embodiments, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

### Definitions

For clarity, certain terms used in the specification are defined and presented as follows:

As used herein and in the appended claims, the singular forms “a,” “an,” and “the” include plural reference unless the context clearly dictates otherwise. Thus, for example, reference to “a plant” is a reference to one or more plants and includes equivalents thereof known to those skilled in the art, and so forth.

As used herein, the word “or” also encompasses “and/or” unless the context clearly indicates otherwise.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). With regard to a temperature the term “about” means±1° C., preferably ±0.5° C. Where the term “about” is used in the context of this disclosure (e.g., in combinations with temperature or molecular weight values) the exact value (i.e., without “about”) is preferred.

As used herein, phrases such as “between about X and Y”, “between about X and about Y”, “from X to Y” and “from about X to about Y” (and similar phrases) should be interpreted to include X and Y, unless the context indicates otherwise.

The terms “comprises”, “comprising”, “includes”, “including”, “having” and their conjugates mean “including but not limited to”. The term “consisting of means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

Units, prefixes and symbols may be denoted in their SI accepted form. Unless otherwise indicated, nucleic acids are written left to right in 5′ to 3′ orientation; amino acid sequences are written left to right in N-terminus to C-terminus orientation, respectively. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Activity” of the insecticidal proteins of the disclosure means that the insecticidal proteins function as orally active pest (e.g. insect) control agents, have a toxic effect (e.g., inhibiting the ability of the insect pest to survive, grow, and/or reproduce), and/or are able to disrupt or deter pest feeding, which may or may not cause death of the insect. When an insecticidal protein of the disclosure is delivered to the pest, the result is typically death of the pest, or the pest does not feed upon the source that makes the insecticidal protein available to the pest.

The term “chimeric polynucleotide” or “chimeric protein” (or similar terms) as used herein refers to a molecule comprising two or more polynucleotides or proteins, or fragments thereof, of different origin assembled into a single molecule. The term “chimeric construct”, “chimeric gene”, “chimeric polynucleotide” or “chimeric nucleic acid” refers to any construct or molecule that contains, without limitation, (1) polynucleotides (e.g., DNA), including regulatory and coding polynucleotides that are not found together in nature (i.e., at least one of the polynucleotides in the construct is heterologous with respect to at least one of its other polynucleotides), or (2) polynucleotides encoding parts of proteins not naturally adjoined, or (3) parts of promoters that are not naturally adjoined. Further, a chimeric construct, chimeric gene, chimeric polynucleotide or chimeric nucleic acid may comprise regulatory polynucleotides and coding polynucleotides that are derived from different sources, or comprise regulatory polynucleotides and coding polynucleotides derived from the same source, but arranged in a manner different from that found in nature. In some embodiments of the disclosure, the chimeric construct, chimeric gene, chimeric polynucleotide or chimeric nucleic acid comprises an expression cassette comprising a polynucleotide of the disclosure under the control of regulatory polynucleotides, particularly under the control of regulatory polynucleotides functional in plants or bacteria. The word “chimeric” and “hybrid,” with respect to a polynucleotide or protein, are used interchangeably herein.

In the context of the present disclosure, a “chimeric” protein is a protein created by fusing all or a portion of at least two different proteins. A chimeric protein may also be further modified to include additions, substitutions and/or deletions of one or more amino acids. In some embodiments of the present disclosure, the chimeric protein is a chimeric Cry protein comprising all or a portion of two different Cry proteins fused together in a single polypeptide. In some embodiments, the chimeric Cry protein further comprises additional modifications such as additions, substitutions, and/or deletions of one or more amino acids. A “chimeric insecticidal protein” is a chimeric protein that has insecticidal activity.

As used herein, a “codon optimized” sequence means a nucleotide sequence wherein the codons are chosen to reflect the particular codon bias that a host cell or organism may have. This is typically done in such a way so as to preserve the amino acid sequence of the polypeptide encoded by the nucleotide sequence to be optimized. In certain embodiments, the DNA sequence of the recombinant DNA construct includes sequence that has been codon optimized for the cell (e.g., an animal, plant, or fungal cell) in which the construct is to be expressed. For example, a construct to be expressed in a plant cell can have all or parts of its sequence (e.g., the first gene suppression element or the gene expression element) codon optimized for expression in a plant. See, for example, U.S. Pat. No. 6,121,014, which is incorporated herein by reference. In some embodiments, the polynucleotides of the disclosure are codon-optimized for expression in a plant cell (e.g., a dicot cell or a monocot cell) or bacterial cell.

To “control” insects means to inhibit, through a toxic effect, the ability of insect pests to survive, grow, feed, and/or reproduce, and/or to limit insect-related damage or loss in crop plants and/or to protect the yield potential of a crop when grown in the presence of insect pests. To “control” insects may or may not mean killing the insects, although in some embodiments of the disclosure, “control” of the insect means killing the insects.

A “control plant” or “control” as used herein may be a non-transgenic plant of the parental line used to generate a transgenic plant herein. A control plant may in some cases be a transgenic plant line that includes an empty vector or marker gene but does not contain the recombinant polynucleotide of the present disclosure that is expressed in the transgenic plant being evaluated. In general, a control plant is a plant of the same line or variety as the transgenic plant being tested, lacking the specific trait-conferring, recombinant DNA that characterizes the transgenic plant. Such a progenitor plant that lacks that specific trait-conferring recombinant DNA can be a natural, wild-type plant, an elite, non-transgenic plant, or a transgenic plant without the specific trait-conferring, recombinant DNA that characterizes the transgenic plant. The progenitor plant lacking the specific, trait-conferring recombinant DNA can be a sibling of a transgenic plant having the specific, trait-conferring recombinant DNA. Such a progenitor sibling plant may include other recombinant DNA.

In the context of the disclosure, “corresponding to” or “corresponds to” means that when the amino acid sequences of a reference sequence are aligned with a second amino acid sequence (e.g. variant or homologous sequences), different from the reference sequence, the amino acids that “correspond to” certain enumerated positions in the second amino acid sequence are those that align with these positions in the reference amino acid sequence but that are not necessarily in the exact numerical positions relative to the particular reference amino acid sequence of the disclosure.

As used herein, the term “Cry protein” means an insecticidal protein of a Bacillus thuringiensis crystal delta-endotoxin type. The term “Cry protein” can refer to the protoxin form or any insecticidally active fragment or toxin thereof including partially processed and the mature toxin form (e.g., without the N-terminal peptidyl fragment and/or the C-terminal protoxin tail).

To “deliver” or “delivering” a composition or toxin means that the composition or toxin comes in contact with an insect, resulting in a toxic effect and control of the insect. The composition or toxin can be delivered in many recognized ways, e.g., orally by ingestion by the insect via transgenic plant expression.

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide group.

An “engineered” protein of the disclosure refers to a protein that has a sequence that is different at at least one amino acid position compared to at least one corresponding parent protein. An engineered protein can be a mutant protein that contains, e.g., one or more modifications such as deletions, additions, and/or substitutions of one or more amino acid positions relative to a parent protein. An engineered protein can be a chimeric protein and contain, e.g., one or more swapped or shuffled domains or fragments from at least two parent proteins.

“Effective insect-controlling amount” means that concentration of toxin or toxins that inhibits, through a toxic effect, the ability of insects to survive, grow, feed and/or reproduce, or to limit insect-related damage or loss in crop plants. “Effective insect-controlling amount” may or may not mean killing the insects, although it preferably means killing the insects. “Insecticidal” is defined as a toxic biological activity capable of controlling insects, preferably by killing them. A transgenic plant with “enhanced insecticidal properties” is a plant that is expresses a protein or proteins at effective insect-controlling amounts, so that, in some embodiments, the plant is insecticidal to an increased range of insect species, relative to a plant of the same kind which is not transformed. This increased range of insect species includes insect plant pests, such as lepidopteran insect pests, e.g., Spodoptera frugiperda (fall armyworm).

The term “event” refers to the original transformant and/or progeny of the transformant that include the heterologous DNA. The term “event” also refers to progeny produced by a sexual outcross between the transformant and another maize line. Even after repeated backcrossing to a recurrent parent, the inserted DNA and the flanking DNA from the transformed parent is present in the progeny of the cross at the same chromosomal location. The term “event” also refers to DNA from the original transformant comprising the inserted DNA and flanking genomic sequence immediately adjacent to the inserted DNA that would be expected to be transferred to a progeny as the result of a sexual cross of one parental line that includes the inserted DNA (e.g., the original transformant and progeny resulting from selfing) and a parental line that does not contain the inserted DNA. Typically, transformation of plant tissue produces multiple events, each of which represent insertion of a DNA construct into a different location in the genome of a plant cell.

“Expression cassette” as used herein means a nucleic acid sequence capable of directing expression of a particular nucleotide sequence(s) in an appropriate host cell, comprising one or more transgene, each transgene comprising a promoter operably linked to a nucleotide sequence of interest which is operably linked to termination signals. Each transgene also typically comprises sequences required for proper translation of the nucleotide sequence. The expression cassette comprising the nucleotide sequence(s) of interest may have at least one of its components heterologous with respect to at least one of its other components. The expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter that initiates transcription only when the host cell is exposed to some particular external stimulus. In the case of a multicellular organism, such as a plant, the promoter can also be specific to a particular tissue, or organ, or stage of development.

An expression cassette comprising a nucleotide sequence(s) of interest may be chimeric, meaning that at least one of its components is heterologous with respect to at least one of its other components. An expression cassette may also be one that comprises a native promoter driving its native gene; however, it has been obtained in a recombinant form useful for heterologous expression. Such usage of an expression cassette makes it so it is not naturally occurring in the cell into which it has been introduced.

An expression cassette also can optionally include one or more transcriptional and/or translational termination regions that is/are functional in plants. A variety of transcriptional terminators are available for use in expression cassettes and are responsible for the termination of transcription beyond the heterologous nucleotide sequence of interest and correct mRNA polyadenylation. The termination region may be native to the transcriptional initiation region, may be native to the operably linked nucleotide sequence of interest, may be native to the plant host, or may be derived from another source (i.e., foreign or heterologous to the promoter, the nucleotide sequence of interest, the plant host, or any combination thereof).

A “gene” comprises a coding nucleic acid sequence and typically also comprises other, primarily regulatory, nucleic acids responsible for the control of the expression, that is to say the transcription and translation, of the coding portion. A gene may also comprise other 5′ and 3′ untranslated sequences and termination sequences. Further elements that may be present are, for example, introns. The regulatory nucleic acid sequence of the gene may not normally be operatively linked to the associated nucleic acid sequence as found in nature and thus would be a chimeric gene.

The term “germplasm” refers to genetic material of or from an individual (e.g., a plant), a group of individuals (e.g., a plant line, variety or family), or a clone derived from a line, variety, species, or culture. The germplasm can be part of an organism or cell or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leaves, stems, pollen, or cells, which can be cultured into a whole plant.

The term “heterologous” when used in reference to a gene or a polynucleotide or a polypeptide refers to a gene or a polynucleotide or a polypeptide that is or contains a part thereof not in its natural environment (i.e., has been altered by the hand of man). For example, a heterologous gene may include a polynucleotide from one species introduced into another species. A heterologous gene may also include a polynucleotide native to an organism that has been altered in some way (e.g., mutated, added in multiple copies, linked to a non-native promoter or enhancer polynucleotide, etc.). Heterologous genes further may comprise plant gene polynucleotides that comprise cDNA forms of a plant gene; the cDNAs may be expressed in either a sense (to produce mRNA) or anti-sense orientation (to produce an anti-sense RNA transcript that is complementary to the mRNA transcript). In one aspect of the disclosure, heterologous genes are distinguished from endogenous plant genes in that the heterologous gene polynucleotides are typically joined to polynucleotides comprising regulatory elements such as promoters that are not found naturally associated with the gene for the protein encoded by the heterologous gene or with plant gene polynucleotides in the chromosome, or are associated with portions of the chromosome not found in nature (e.g., genes expressed in loci where the gene is not normally expressed). Further, a “heterologous” polynucleotide refers to a polynucleotide not naturally associated with a host cell into which it is introduced, including non-naturally occurring multiple copies of a naturally occurring polynucleotide.

The terms “increase”, “increasing”, “increased”, “enhance”, “enhanced”, “enhancing”, and “enhancement” and similar terms, as used herein, describe an elevation in control of a plant pest, e.g., by contacting pest with a plant of the disclosure (such as, for example, by transgenic expression or by topical application methods). The increase in control can be in reference to the level of control of the plant pest in the absence of a nucleic acid molecule of the disclosure (e.g., a plant that does not comprise the nucleic acid molecule). Thus in embodiments, the terms “increase”, “increasing”, “increased”, “enhance”, “enhanced”, “enhancing”, and “enhancement” and similar terms can indicate an elevation of at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 200%, 300%, 400%, 500% or more as compared to a suitable control (e.g., a plant, plant part, plant cell that does not comprise the nucleic acid molecule).

The term “identity” or “identical” in the context of two nucleic acid or amino acid sequences, refers to the percentage of identical nucleotides or amino acids in a linear polynucleotide or amino acid sequence of a reference (“query”) sequence (or its complementary strand) as compared to a test (“subject”) sequence when the two sequences are globally aligned. Unless otherwise stated, sequence identity as used herein refers to the value obtained using the Needleman and Wunsch algorithm ((1970) J. Mol. Biol. 48:443-453) implemented in the EMBOSS Needle alignment tool using default matrix files EBLOSUM62 for protein with default parameters (Gap Open=10, Gap Extend=0.5, End Gap Penalty=False, End Gap Open=10, End Gap Extend=0.5) or DNA full for nucleic acids with default parameters (Gap Open=10, Gap Extend=0.5, End Gap Penalty=False, End Gap Open=10, End Gap Extend=0.5); or any equivalent program thereof. EMBOSS Needle is available, e.g., from EMBL-EBI such as at the following website: ebi.ac.uk/Tools/psa/emboss_needle/ and as described in the following publication: “The EMBL-EBI search and sequence analysis tools APIs in 2019.” Madeira et al. Nucleic Acids Research, June 2019, 47(W1):W636-W641. The term “equivalent program” as used herein refers to any sequence comparison program that, for any two sequences in question, generates an alignment having identical nucleotide or amino acid residue matches and an identical percent sequence identity when compared to the corresponding alignment generated by EMBOSS Needle. In some embodiments, substantially identical nucleic acid or amino acid sequences may perform substantially the same function.

In some embodiments, the polynucleotides or polypeptides of the disclosure are “isolated”. The term “isolated” polynucleotide or polypeptide is a polynucleotide or polypeptide that no longer exists in its natural environment. An isolated polynucleotide or polypeptide of the disclosure may exist in a purified form or may exist in a recombinant host such as in a transgenic bacteria or a transgenic plant. Therefore, in some embodiments, an “isolated” nucleic acid molecule encompasses a nucleic acid molecule when the nucleic acid molecule is comprised within a transgenic plant genome.

The term “isolated”, when used in the context of the nucleic acid molecules or polynucleotides of the present disclosure, refers to a polynucleotide that is identified within and isolated/separated from its chromosomal polynucleotide context within the respective source organism. An isolated nucleic acid or polynucleotide is not a nucleic acid as it occurs in its natural context, if it indeed has a naturally occurring counterpart. In contrast, non-isolated nucleic acids are nucleic acids such as DNA and RNA, which are found in the state they exist in nature. For example, a given polynucleotide (e.g., a gene) is found on the host cell chromosome in proximity to neighboring genes. The isolated nucleic acid molecule may be present in single-stranded or double-stranded form. Alternatively, it may contain both the sense and antisense strands (i.e., the nucleic acid molecule may be double-stranded). In some embodiments, the nucleic acid molecules of the present disclosure are isolated.

As used herein, the term “maize” includes Zea mays and all plant varieties that can be bred with Zea mays, including wild maize species. The term “maize” and “com” are used interchangeably herein.

The term “motif” or “consensus sequence” or “signature” refers to a short conserved region in the sequence of evolutionarily related proteins. Motifs are frequently highly conserved parts of domains, but may also include only part of the domain, or be located outside of conserved domain (if all of the amino acids of the motif fall outside of a defined domain).

A “native” or “wild type” nucleic acid, polynucleotide, nucleotide sequence, polypeptide or amino acid sequence refers to a naturally occurring or endogenous nucleic acid, polynucleotide, nucleotide sequence, polypeptide or amino acid sequence.

A “nucleic acid molecule” or “nucleic acid” or “polynucleotide” (which are used interchangeably herein) is a segment of single-stranded, double-stranded or partially double-stranded DNA or RNA, or a hybrid thereof, that can be isolated or synthesized from any source. In the context of the present disclosure, the nucleic acid molecule is typically a segment of DNA. In some embodiments, the nucleic acid molecules of the disclosure are isolated nucleic acid molecules. In some embodiments, the nucleic acid molecules of the disclosure are comprised within a vector, a plant, a plant cell or a bacterial cell. The terms also include reference to a deoxyribopolynucleotide, ribopolynucleotide or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A nucleic acid molecule can be full-length or a subsequence of a native or heterologous structural or regulatory gene. Unless otherwise indicated, the term includes reference to the specified sequence as well as the complementary sequence thereof.

Thus, DNAs or RNAs with backbones modified for stability or for other reasons are “polynucleotides” as that term is intended herein. Moreover, DNAs or RNAs comprising unusual bases, such as inosine or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

“Operably linked” refers to the association of polynucleotides on a single nucleic acid molecule so that the function of one affects the function of the other. For example, a promoter is operably linked with a coding polynucleotide when it is capable of affecting the expression of that coding polynucleotide (i.e., that the coding polynucleotide is under the transcriptional control of the promoter). Coding polynucleotide in sense or antisense orientation can be operably linked to regulatory polynucleotides.

The term “plant” includes reference to whole plants, plant organs, plant tissues (e.g., leaves, stems, roots, etc.), seeds and plant cells and progeny of same. Plant cell, as used herein includes, without limitation, seeds, suspension cultures, embryos, meristematic regions, callus tissue, leaves, roots, shoots, gametophytes, sporophytes, pollen and microspores. The class of plants, which can be used in the methods of the disclosure, is generally as broad as the class of higher plants amenable to transformation techniques, including both monocotyledonous and dicotyledonous plants including species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, Allium and Triticum. A particularly preferred plant is maize.

A “plant cell” is a structural and physiological unit of a plant, comprising a protoplast and a cell wall. The plant cell may be in the form of an isolated single cell or a cultured cell, or as a part of a higher organized unit such as, for example, plant tissue, a plant organ, or a whole plant.

“Plant cell culture” means cultures of plant units such as, for example, protoplasts, cell culture cells, cells in plant tissues, pollen, pollen tubes, ovules, embryo sacs, zygotes and embryos at various stages of development.

“Plant material” refers to leaves, stems, roots, flowers or flower parts, fruits, pollen, egg cells, zygotes, seeds, cuttings, cell or tissue cultures, or any other part or product of a plant.

A “plant organ” is a distinct and visibly structured and differentiated part of a plant such as a root, stem, leaf, flower bud, or embryo.

As used herein, “plant material,” “plant part” or “plant tissue” means plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, tubers, rhizomes and the like. Any tissue of a plant in planta or in culture is included in the term “plant tissue.”

As used herein “plant sample” or “biological sample” refers to either intact or non-intact (e.g. milled seed or plant tissue, chopped plant tissue, lyophilized tissue) plant tissue. It may also be an extract comprising intact or non-intact seed or plant tissue. The biological sample or extract may be selected from the group consisting of corn flour, corn meal, corn syrup, corn oil, corn starch, and cereals manufactured in whole or in part to contain corn by-products.

A “polynucleotide of interest” or “nucleic acid of interest” refers to any polynucleotide which, when transferred to an organism, e.g., a plant, confers upon the organism a desired characteristic such as insect resistance, disease resistance, herbicide tolerance, antibiotic resistance, improved nutritional value, improved performance in an industrial process, production of a commercially valuable enzyme or metabolite, an altered reproductive capability, and the like.

A “portion” or a “fragment” of a polypeptide of the disclosure will be understood to mean an amino acid sequence or nucleic acid sequence of reduced length relative to a reference amino acid sequence or nucleic acid sequence of the disclosure. Such a portion or a fragment according to the disclosure may be, where appropriate, included in a larger polypeptide or nucleic acid of which it is a constituent (e.g., a tagged or fusion protein or an expression cassette). In embodiments, the “portion” or “fragment” substantially retains the activity, such as insecticidal activity (e.g., at least 40%, 50%, 60%, 70%, 80%, 85%, 90%, 95% or even 100% of the activity) of the full-length protein or nucleic acid, or has even greater activity, e.g., insecticidal activity, than the full-length protein).

As used herein, “propagule” refers to any material that is used for propagating a plant, preferably a transgenic plant. A propagule may be a seed, cutting, or plurality of cells from a transgenic plant, which can be used to produce a crop of transgenic plants.

The terms “protein,” “peptide,” and “polypeptide” are used interchangeably herein.

The term “promoter,” as used herein, refers to a polynucleotide, usually upstream (5′) of the translation start site of a coding sequence, which controls the expression of the coding sequence by providing the recognition for RNA polymerase and other factors required for proper transcription. For example, a promoter may contain a region containing basal promoter elements recognized by RNA polymerase, a region containing the 5′ untranslated region (UTR) of a coding sequence, and optionally an intron.

A “pollen-free promoter” is a promoter that drives low or no detectable gene expression in the pollen of the target plant species. Quantification of mRNA transcripts of a protein of interest in pollen could be measured by various methods including qRT-PCR/RNA-Seq; the protein can be measured by commonly used ELISA and Western blot methodology. A promoter is considered pollen-free in this disclosure if the promoter drives expression of a protein of the disclosure at <10 ng/mg TSP (total soluble protein) in pollen.

As used herein, the term “recombinant” refers to a form of nucleic acid (e.g., DNA or RNA), protein, cell, tissue, organism and the like that would not normally be found in nature and as such was created by human intervention. As used herein, a “recombinant nucleic acid molecule” is a nucleic acid molecule comprising a combination of polynucleotides that would not naturally occur together and is the result of human intervention, e.g., a nucleic acid molecule that is comprised of a combination of at least two polynucleotides heterologous to each other, or a nucleic acid molecule that is artificially synthesized, for example, a polynucleotide synthesized using an assembled nucleotide sequence, and comprises a polynucleotide that deviates from the polynucleotide that would normally exist in nature, or a nucleic acid molecule that comprises a transgene artificially incorporated into a host cell's genomic DNA and the associated flanking DNA of the host cell's genome. Another example of a recombinant nucleic acid molecule is a DNA molecule resulting from the insertion of a transgene into a plant's genomic DNA, which may ultimately result in the expression of a recombinant RNA or protein molecule in that organism. As used herein, a “recombinant plant” is a plant that would not normally exist in nature, is the result of human intervention, and contains a transgene or heterologous nucleic acid molecule which may be incorporated into its genome. As a result of such genomic alteration, the recombinant plant is distinctly different from the related wild-type plant. A “recombinant” bacteria is a bacteria not found in nature that comprises a heterologous nucleic acid molecule. Such a bacteria may be created by transforming the bacteria with the nucleic acid molecule or by the conjugation-like transfer of a plasmid from one bacteria strain to another, whereby the plasmid comprises the nucleic acid molecule.

The terms “reduce,” “reduced,” “reducing,” “reduction,” “diminish,” and “suppress” (and grammatical variations thereof) and similar terms, as used herein, refer to a decrease in the survival, growth and/or reproduction of a plant pest, e.g., by contacting a pest with a plant of the disclosure. This decrease in survival, growth and/or reproduction can be in reference to the level observed in the absence of a nucleic acid molecule of the disclosure (e.g., a plant that does not comprise the nucleic acid molecule). Thus, in embodiments, the terms “reduce,” “reduced,” “reducing,” “reduction,” “diminish,” and “suppress” (and grammatical variations thereof) and similar terms mean a decrease of at least about 5%, 10%, 15%, 20%, 25%, 30%, 35%, 40%, 45%, 50%, 55%, 60%, 65%, 70%, 75%, 80%, 85%, 90%, 95% or more as compared with a plant that is not contacted with a nucleic acid molecule of the disclosure (e.g., a plant that does not comprise the nucleic acid molecule). In representative embodiments, the reduction results in no or essentially no (i.e., an insignificant amount, e.g., less than about 10%, less than about 5% or even less than about 1%) detectable survival, growth and/or reproduction of the plant pest.

“Regulatory elements” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translational enhancer sequences, introns, terminators, and polyadenylation signal sequences. They include natural and synthetic sequences as well as sequences which may be a combination of synthetic and natural sequences. Regulatory sequences may determine expression level, the spatial and temporal pattern of expression and, for a subset of promoters, expression under inductive conditions (regulation by external factors such as light, temperature, chemicals and hormones).

As used herein, “selectable marker” means a nucleotide sequence that when expressed imparts a distinct phenotype to the plant, plant part and/or plant cell expressing the marker and thus allows such transformed plants, plant parts and/or plant cells to be distinguished from those that do not have the marker. Such a nucleotide sequence may encode either a selectable or screenable marker, depending on whether the marker confers a trait that can be selected for by chemical means, such as by using a selective agent (e.g., an antibiotic, herbicide, or the like), or on whether the marker is simply a trait that one can identify through observation or testing, such as by screening (e.g., the R-locus trait).

The terms “stringent conditions” or “stringent hybridization conditions” include reference to conditions under which a nucleic acid will selectively hybridize to a target sequence to a detectably greater degree than other sequences (e.g., at least 2-fold over a non-target sequence), and optionally may substantially exclude binding to non-target sequences.

Stringent conditions are sequence-dependent and will vary under different circumstances. By controlling the stringency of the hybridization and/or washing conditions, target sequences can be identified that can be up to 100% complementary to the reference nucleotide sequence. Alternatively, conditions of moderate or even low stringency can be used to allow some mismatching in sequences so that lower degrees of sequence similarity are detected. For example, those skilled in the art will appreciate that to function as a primer or probe, a nucleic acid sequence only needs to be sufficiently complementary to the target sequence to substantially bind thereto so as to form a stable double-stranded structure under the conditions employed. Thus, primers or probes can be used under conditions of high, moderate or even low stringency. Likewise, conditions of low or moderate stringency can be advantageous to detect homolog, ortholog and/or paralog sequences having lower degrees of sequence identity than would be identified under highly stringent conditions. Typically, stringent conditions are those in which the salt concentration is less than about 1.5 M Na ion, typically about 0.01 to 1.0 M Na ion concentration (or other salts) at about pH 7.0 to pH 8.3 and the temperature is at least about 30° C. for short probes (e.g., 10 to 50 nucleotides) and at least about 60° C. for longer probes (e.g., greater than 50 nucleotides). Stringent conditions may also be achieved with the addition of destabilizing agents such as formamide or Denhardt's (5 g Ficoll, 5 g polyvinylpyrrolidone, 5 g bovine serum albumin in 500 ml of water). Exemplary low stringency conditions include hybridization with a buffer solution of 30% to 35% formamide, 1 M NaCl, 1% SDS (sodium dodecyl sulfate) at 3TC and a wash in 1× to 2×SSC (20×SSC=3.0 M NaCl/0.3 M trisodium citrate) at 50° C. to 55° C. Exemplary moderate stringency conditions include hybridization in 40% to 45% formamide, 1 M NaCl, 1% SDS at 37° C. and a wash in 0.5× to 1×SSC at 55° C. to 60° C. Exemplary high stringency conditions include hybridization in 50% formamide, 1 M NaCl, 1% SDS at 3TC and a wash in 0.1×SSC at 60° C. to 65° C. A further non-limiting example of high stringency conditions include hybridization in 4×SSC, 5×Denhardt's, 0.1 mg/ml boiled salmon sperm DNA, and 25 mM Na phosphate at 65° C. and a wash in 0.1×SSC, 0.1% SDS at 65° C. Another illustration of high stringency hybridization conditions includes hybridization in 7% SDS, 0.5 M NaPO4, 1 mM EDTA at 50° C. with washing in 2×SSC, 0.1% SDS at 50° C., alternatively with washing in 1×SSC, 0.1% SDS at 50° C., alternatively with washing in 0.5×SSC, 0.1% SDS at 50° C., or alternatively with washing in 0.1×SSC, 0.1% SDS at 50° C., or even with washing in 0.1×SSC, 0.1% SDS at 65° C. Those skilled in the art will appreciate that specificity is typically a function of post-hybridization washes, the relevant factors being the ionic strength and temperature of the final wash solution.

“Stable transformation” or “stably transformed” as used herein means that a nucleic acid is introduced into a cell and integrates into the genome of the cell. As such, the integrated nucleic acid is capable of being inherited by the progeny thereof, more particularly, by the progeny of multiple successive generations. “Genome” as used herein also includes the nuclear and the plastid genome, and therefore includes integration of the nucleic acid into, for example, the chloroplast genome. Stable transformation as used herein can also refer to a transgene that is maintained extrachromasomally, for example, as a minichromosome.

As used herein, gene or trait “stacking” is combining desired genes or traits into one transgenic plant line. As one approach, plant breeders stack transgenic traits by making crosses between parents that each have a desired trait and then identifying offspring that have both of these desired traits (so-called “breeding stacks”). Another way to stack genes is by transferring two or more genes into the cell nucleus of a plant at the same time during transformation. Another way to stack genes is by re-transforming a transgenic plant with another gene of interest. For example, gene stacking can be used to combine two different insect resistance traits, an insect resistance trait and a disease resistance trait, or a herbicide resistance trait (such as, for example, Bt11). The use of a selectable marker in addition to a gene of interest would also be considered gene stacking.

“Synthetic” refers to a nucleotide sequence comprising bases or a structural feature(s) that is not present in the natural sequence. For example, an artificial sequence encoding a protein of the disclosure that resembles more closely the G+C content and the normal codon distribution of dicot or monocot plant genes is said to be synthetic.

As used herein, a protein of the disclosure that is “toxic” to an insect pest is meant that the protein functions as an orally active insect control agent to kill the insect pest, or the protein is able to disrupt or deter insect feeding, or causes growth inhibition to the insect pest, both of which may or may not cause death of the insect. When a toxic protein of the disclosure is delivered to an insect or an insect comes into oral contact with the toxic protein, the result is typically death of the insect, or the insect's growth is slowed, or the insect stops feeding upon the source that makes the toxic protein available to the insect.

The terms “toxin fragment” and “toxin portion” are used interchangeably herein to refer to a fragment or portion of a longer (e.g., full-length) insecticidal protein of the disclosure, where the “toxin fragment” or “toxin portion” retains insecticidal activity. For example, it is known in the art that native Cry proteins are expressed as protoxins that are processed at the N-terminal and C-terminal ends to produce a mature toxin. In embodiments, the “toxin fragment” or “toxin portion” of a chimeric insecticidal protein of the disclosure is truncated at the N-terminus and/or C-terminus. In embodiments, the “toxin fragment” or “toxin portion” is truncated at the N-terminus to remove part or all of the N-terminal peptidyl fragment, and optionally comprises at least about 400, 425, 450, 475, 500, 510, 520, 530, 540, 550, 560, 570, 580 or 590 contiguous amino acids of insecticidal protein specifically described herein or an amino acid sequence that is substantially identical thereto. Thus, in embodiments, a “toxin fragment” or “toxin portion” of an insecticidal protein is truncated at the N-terminus (e.g., to omit part or all of the peptidyl fragment), for example, an N-terminal truncation of one amino acid or more than one amino acid, e.g., up to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more amino acids. In embodiments, a “toxin fragment” or “toxin portion” of an insecticidal protein is truncated at the C-terminus (e.g., to omit part or all of the protoxin tail), for example, a C-terminal truncation of one amino acid or more than one amino acid, e.g., up to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, 100, 125, 150, 175, 200, 225, 250, 275, 300, 325, 350, 375, 400, 425, 450, 475, 500, 525, 550, 560 or more amino acids. In embodiments, the “toxin fragment” or “toxin portion” comprises domains 1 and 2, and the core domain 3. In embodiments, the “toxin fragment” or “toxin portion” is the mature (i.e., processed) toxin (e.g., Cry toxin).

“Transformation” is a process for introducing heterologous nucleic acid into a host cell or organism. In particular embodiments, “transformation” means the stable integration of a DNA molecule into the genome (nuclear or plastid) of an organism of interest. In some particular embodiments, the introduction into a plant, plant part and/or plant cell is via bacterial-mediated transformation, particle bombardment transformation, calcium-phosphate-mediated transformation, cyclodextrin-mediated transformation, electroporation, liposome-mediated transformation, nanoparticle-mediated transformation, polymer-mediated transformation, virus-mediated nucleic acid delivery, whisker-mediated nucleic acid delivery, microinjection, sonication, infiltration, polyethylene glycol-mediated transformation, protoplast transformation, or any other electrical, chemical, physical and/or biological mechanism that results in the introduction of nucleic acid into the plant, plant part and/or cell thereof, or a combination thereof. Procedures for transforming plants are well known and routine in the art and are described throughout the literature. Non-limiting examples of methods for transformation of plants include transformation via bacterial-mediated nucleic acid delivery (e.g., via bacteria from the genus Agrobacterium), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (2002, Cell Mol Biol Lett 7:849-858 (2002)).

“Transformed” and “transgenic” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed”, “non-transgenic”, or “non-recombinant” host refers to a wild-type organism, e.g., a bacterium or plant, which does not contain the heterologous nucleic acid molecule.

The term “transgenic plant” includes reference to a plant into which a heterologous nucleic acid molecule has been introduced. Generally, the heterologous nucleic acid sequence is stably integrated within the genome such that the nucleic acid sequence is passed on to successive generations. The heterologous nucleic acid sequence may be integrated into the genome alone or as part of a recombinant expression cassette. “Transgenic” is used herein to include any cell, cell line, callus, tissue, plant part or plant, the genotype of which has been altered by the presence of a heterologous nucleic acid sequence, including those transgenics initially so altered as well as those created by sexual crosses or asexual propagation from the initial transgenic.

The term “vector” refers to a composition for transferring, delivering or introducing a nucleic acid (or nucleic acids) into a cell. A vector comprises a nucleic acid molecule comprising the nucleotide sequence(s) to be transferred, delivered or introduced. Example vectors include a plasmid, cosmid, phagemid, artificial chromosome, phage or viral vector.

The term “yield” may include reference to bushels per acre of a grain crop at harvest, as adjusted for grain moisture (15% typically for corn, for example), and the volume of biomass generated (for forage crops such as alfalfa and plant root size for multiple crops). Grain moisture is measured in the grain at harvest. The adjusted test weight of grain is determined to be the weight in pounds per bushel, adjusted for grain moisture level at harvest. Biomass is measured as the weight of harvestable plant material generated. Yield can be affected by many properties including without limitation, plant height, pod number, pod position on the plant, number of internodes, incidence of pod shatter, grain size, efficiency of nodulation and nitrogen fixation, efficiency of nutrient assimilation, carbon assimilation, plant architecture, percent seed germination, seedling vigor, and juvenile traits. Yield can also be affected by efficiency of germination (including germination in stressed conditions), growth rate (including growth rate in stressed conditions), ear number, seed number per ear, seed size, composition of seed (starch, oil, protein) and characteristics of seed fill. Yield of a plant of the can be measured in a number of ways, including test weight, seed number per plant, seed weight, seed number per unit area (i.e. seeds, or weight of seeds, per acre), bushels per acre, tons per acre, or kilo per hectare. For example, corn yield may be measured as production of shelled corn kernels per unit of production area, for example in bushels per acre or metric tons per hectare, often reported on a moisture adjusted basis, for example at 15.5 percent moisture. Moreover a bushel of corn is defined by law in the State of Iowa as 56 pounds by weight, a useful conversion factor for corn yield is: 100 bushels per acre is equivalent to 6.272 metric tons per hectare. Other measurements for yield are common practice in the art. In certain embodiments of the disclosure yield may be increased in stressed and/or non-stressed conditions.

### Nucleic Acid Molecules

The present disclosure provides compositions and methods for controlling harmful plant pests. Particularly, the present disclosure provides a nucleic acid molecule that, when expressed in a cell, confers insecticidal properties on the cell, e.g., insecticidal activity against Lepidopteran pests such as Spodoptera frugiperda (fall armyworm).

Multiple different constructs were produced to determine the efficacy and agronomic impact of the expressed protein(s) in the context of different expression cassettes. Surprisingly, one vector, SEQ ID NO: 2, when transformed into maize plants conferred excellent insecticidal properties with no or minimal negative effects on the vegetative development or the fertility of the transgenic plant. The expression cassette from the vector is SEQ ID NO: 1.

A skilled person would recognize that during the insertion of a nucleic acid molecule, such as SEQ ID NO: 1, into a cell, the 5′ and/or 3′ ends of the inserted molecule may be deleted or rearranged. Such deletions or rearrangements may not affect the function of the inserted molecule, and these relatively small changes result in an inserted molecule that may be considered to be substantially identical to SEQ ID NO: 1. A skilled person would also recognize that the nucleic acid molecule, such as one comprising SEQ ID NO: 1, may undergo full or partial rearrangement or duplication during the insertion event, such that the inserted molecule is a full or partial rearrangement or duplication of the starting nucleic acid molecule. A skilled person would recognize that this inserted molecule may still have the same characteristics and/or traits as the starting molecule, such that the inserted molecule is substantially identical to SEQ ID NO: 1, and the transformed cell or resulting transformed plant may still be desirable.

A skilled person would recognize that a transgene for commercial use, such as a nucleic acid molecule that comprises SEQ ID NO: 1, may need relatively minor modifications to the nucleic acid sequence to comply with governmental regulatory standards. Such modifications should not affect the function of the resulting molecule, which would be substantially identical to SEQ ID NO: 1. A skilled person would recognize that the modified nucleic acid molecule would be essentially the same as the starting molecule.

Therefore, the disclosure also encompasses a nucleic acid molecule substantially identical to SEQ ID NO: 1, wherein certain nucleotides of SEQ ID NO: 1 are deleted, substituted or rearranged, resulting in a mutated SEQ ID NO:1 and wherein the functionality of the mutated SEQ ID NO:1 is the same as the starting molecule. Accordingly, in some aspects, the disclosure provides a nucleic acid molecule comprising a nucleic acid sequence that is at least 90% identical to SEQ ID NO: 1 (e.g., at least 90% identical to SEQ ID NO: 1, at least 91% identical to SEQ ID NO: 1, at least 92% identical to SEQ ID NO: 1, at least 93% identical to SEQ ID NO: 1, at least 94% identical to SEQ ID NO: 1, at least 95% identical to SEQ ID NO: 1, at least 96% identical to SEQ ID NO: 1, at least 97% identical to SEQ ID NO: 1, at least 98% identical to SEQ ID NO: 1, at least 99% identical to SEQ ID NO: 1, or at least 99.5% identical to SEQ ID NO: 1), or the complement thereof. In some embodiments, the nucleic acid molecule encodes the same protein(s) that are encoded by SEQ ID NO: 1. In some embodiments, the nucleic acid sequence comprises any one of SEQ ID NOs: 1 or 8 to 31 or any one or more of the variants in Table 3. In some embodiments, the nucleic acid molecule produces protein(s) that is/are insecticidal against one or more Lepidopteran pests, e.g., insecticidal against at least Spodoptera frugiperda (fall armyworm). In some embodiments, the nucleic acid molecule produces protein(s) that is/are insecticidal against at least two (e.g., 2, 3, or 4) of Spodoptera frugiperda (fall armyworm), Mythimna separata (oriental armyworm), Spodoptera litura (common cutworm/oriental leafworm), and Ostrinia furnacalis (Asian corn borer). In some embodiments, the nucleic acid molecule is isolated. In some embodiments, the nucleic acid molecule is present in a plant.

The disclosed insecticidal protein(s) encoded by a nucleic acid molecule of the disclosure (e.g., any one of SEQ ID NOs: 1 or 8 to 31 or any one or more of the variants in Table 3) have insecticidal activity against Lepidopteran pests. In some embodiments, the insecticidal protein(s) has/have activity against one or more of the following non-limiting examples of a Lepidopteran pest: Spodoptera spp. such as S. frugiperda (fall armyworm), S. littoralis (Egyptian cotton leafworm), S. ornithogalli (yellowstriped armyworm), S. praefica (western yellowstriped armyworm), S. eridania (southern armyworm), S. litura (Common cutworm/Oriental leafworm), S. cosmioides (black armyworm), S. exempta (African armyworm), S. mauritia (lawn armyworm) and/or S. exigua (beet armyworm); Ostrinia spp. such as O. nubilalis (European corn borer) and/or O. furnacalis (Asian corn borer); Plutella spp. such as P. xylostella (diamondback moth); Agrotis spp. such as A. ipsilon (black cutworm), A. segetum (common cutworm), A. gladiaria (claybacked cutworm), and/or A. orthogonia (pale western cutworm); Striacosta spp. such as S. albicosta (western bean cutworm); Helicoverpa spp. such as H. zea (corn earworm/soybean podworm), H. punctigera (native budworm), and/or H. armigera (cotton bollworm); Heliothis spp. such as H. virescens (tobacco budworm); Diatraea spp. such as D. grandiosella (southwestern corn borer) and/or D. saccharalis (sugarcane borer); Trichoplusia spp. such as T. ni (cabbage looper); Sesamia spp. such as S. nonagroides (Mediterranean corn borer), S. inferens (Pink stem borer) and/or S. calamistis (pink stem borer); Pectinophora spp. such as P. gossypiella (pink bollworm); Cochylis spp. such as C. hospes (banded sunflower moth); Manduca spp. such as M. sexta (tobacco hornworm) and/or M. quinquemaculata (tomato hornworm); Elasmopalpus spp. such as E. lignosellus (lesser cornstalk borer); Pseudoplusia spp. such as P. includens (soybean looper); Anticarsia spp. such as A. gemmatalis (velvetbean caterpillar); Plathypena spp. such as P. scabra (green cloverworm); Pieris spp. such as P. brassicae (cabbage butterfly), Papaipema spp. such as P. nebris (stalk borer); Pseudaletia spp. such as P. unipuncta (common armyworm); Peridroma spp. such as P. saucia (variegated cutworm); Keiferia spp. such as K. lycopersicella (tomato pinworm); Artogeia spp. such as A. rapae (imported cabbageworm); Phthorimaea spp. such as P. operculella (potato tuberworm); Chrysodeixis spp. such as C. includens (soybean looper); Feltia spp. such as F. ducens (dingy cutworm); Chilo spp. such as C. suppressalis (striped stem borer), C. Agamemnon (oriental corn borer), and C. partellus (spotted stalk borer), Cnaphalocrocis spp. such as C. medinalis (rice leaffolder), Conogethes spp. such as C. punctiferalis (Yellow peach moth), Mythimna spp. such as M. separata (Oriental armyworm), Athetis spp. such as A. lepigone (Two-spotted armyworm), Busseola spp. such as B. fusca (maize stalk borer), Etiella spp. such as E. zinckenella (pulse pod borer), Leguminivora spp. such as L. glycinivorella (soybean pod borer), Matsumuraeses spp. such as M. phaseoli (adzuki pod worm), Omiodes spp. such as O. indicata (Soybean leaffolder/Bean-leaf webworm), Rachiplusia spp. such as R. nu (sunflower Looper), or any combination of the foregoing. In some embodiments, at least one of the insecticidal protein(s) encoded by the nucleic acid molecule has/have insecticidal activity against fall armyworm (Spodoptera frugiperda). In some embodiments, at least one of the insecticidal protein(s) encoded by the nucleic acid molecule has/have insecticidal activity against at least two (e.g., 2, 3, or 4) of Spodoptera frugiperda (fall armyworm), Mythimna separata (oriental armyworm), Spodoptera litura (common cutworm/oriental leafworm), and Ostrinia furnacalis (Asian corn borer). In some embodiments, the insecticidal protein(s) can optionally have insecticidal activity against a fall armyworm insect pest or colony that has resistance to another insecticidal agent, including another insecticidal protein (such as, e.g., a Bt protein). In some embodiments, the insecticidal protein(s) has/have insecticidal activity against a fall armyworm colony that is resistant to a Vip3A protein (e.g., a Vip3Aa, including without limitation maize event MIR162), a Cry1F protein (e.g., Cry1Fa, including without limitation maize event TC1507 or DP-4114), a Cry1A protein (e.g., Cry1A.105, including without limitation maize event MON89034), or a Cry2 protein (e.g., Cry2Ab, including without limitation maize event MON89034).

The disclosed insecticidal protein(s) may also have insecticidal activity against Coleopteran, Hemipteran, Dipteran, Lygus spp., and/or other piercing and sucking insects, for example of the order Orthoptera or Thysanoptera. In some embodiments, the insecticidal protein(s) has/have activity against one or more of the following non-limiting examples of a Coleopteran pest: Diabrotica spp. such as D. barberi (northern corn rootworm), D. virgifera virgifera (western corn rootworm), D. undecimpunctata howardii (southern corn rootworm), D. balteata (banded cucumber beetle), D. undecimpunctata undecimpunctata (western spotted cucumber beetle), D. significata (3-spotted leaf beetle), D. speciosa (cucurbit beetle), D. virgifera zeae (Mexican corn rootworm), D. beniensis, D. cristata, D. curviplustalata, D. dissimilis, D. elegantula, D. emorsitans, D. graminea, D. hispanloe, D. lemniscata, D. linsleyi, D. milleri, D. nummularis, D. occlusal, D. porrecea, D. scutellata, D. tibialis, D. trifasciata and/or D. viridula; Leptinotarsa spp. such as L. decemlineata (Colorado potato beetle); Chrysomela spp. such as C. scripta (cottonwood leaf beetle); Hypothenemus spp. such as H. hampei (coffee berry borer); Sitophilus spp. such as S. zeamais (maize weevil); Epitrix spp. such as E. hirtipennis (tobacco flea beetle) and/or E. cucumeris (potato flea beetle); Phyllotreta spp. such as P. cruciferae (crucifer flea beetle) and/or P. pusilla (western black flea beetle); Anthonomus spp. such as A. eugenii (pepper weevil); Hemicrepidus spp. such as H. memnonius (wireworms); Melanotus spp. such as M. communis (wireworm); Ceutorhychus spp. such as C. assimilis (cabbage seedpod weevil); Phyllotreta spp. such as P. cruciferae (crucifer flea beetle); Aeolus spp. such as A. mellillus (wireworm); Aeolus spp. such as A. mancus (wheat wireworm); Horistonotus spp. such as H. uhlerii (sand wireworm); Sphenophorus spp. such as S. maidis (maize billbug), S. zeae (timothy billbug), S. parvulus (bluegrass billbug), and S. callosus (southern corn billbug); Phyllophaga spp. (White grubs); Chaetocnema spp. such as C. pulicaria (corn flea beetle); Popillia spp. such as P. japonica (Japanese beetle); Epilachna spp. such as E. varivestis (Mexican bean beetle); Cerotoma spp. such as C. trifurcate (Bean leaf beetle); Epicauta spp. such as E. pestifera and E. lemniscata (Blister beetles); or any combination of the foregoing. Insects of the order Hemiptera include but are not limited to Chinavia hilaris (green stink bug); Anasa tristis De Geer (squash bug); Blissus leucopterus (chinch bug); Corythuca gossypii Fabricius (cotton lace bug); Cyrtopeltis modesta Distant (tomato bug); Dysdercus suturellus Hern ch-Schaffer (cotton stainer); Euschistus servus Say (brown stink bug); E. variolarius Palisot de Beauvois (one-spotted stink bug); Graptostethus spp. (complex of seed bugs); Leptoglossus corculus Say (leaf-footed pine seed bug); Lygus lineolaris Palisot de Beauvois (tarnished plant bug); L. Hesperus Knight (Western tarnished plant bug); L. pratensis Linnaeus (common meadow bug); L. rugulipennis Poppius (European tarnished plant bug); Lygocoris pabulinus Linnaeus (common green capsid); Nezara viridula Linnaeus (southern green stink bug); Oebalus pugnax Fabricius (rice stink bug); Oncopeltus fasciatus Dallas (large milkweed bug); Pseudatomoscelis seriatus Reuter (cotton fleahopper), Calocoris norvegicus Gmelin (strawberry bug); Orthops campestris Linnaeus; Plesiocoris rugicollis Fallen (apple capsid); Cyrtopeltis modestus Distant (tomato bug); Cyrtopeltis notatus Distant (suckfly); Spanagonicus albofasciatus Reuter (whitemarked fleahopper); Diaphnocoris chlorionis Say (honeylocust plant bug); Labopidicola allii Knight (onion plant bug); Pseudatomoscelis seriatus Reuter (cotton fleahopper); Adelphocoris rapidus Say (rapid plant bug); Poecilocapsus lineatus Fabricius (four-lined plant bug); Nysius ericae Schilling (false chinch bug); Nysius raphanus Howard (false chinch bug); Nezara viridula Linnaeus (Southern green stink bug); Eurygaster spp.; Coreidae spp.; Pyrrhocoridae spp.; Tinidae spp.; Blostomatidae spp.; Reduviidae spp. and Cimicidae spp. Insects in the order Diptera include but are not limited Liriomyza spp. such as L. trifolii (leafminer) and L. sativae (vegetable leafminer); Scrobipalpula spp. such as S. absoluta (tomato leafminer); Delia spp. such as D. platura (seedcom maggot), D. brassicae (cabbage maggot) and D. radicum (cabbage root fly); Psilia spp. such as P. rosae (carrot rust fly); Tetanops spp. such as T. myopaeformis (sugarbeet root maggot); and any combination of the foregoing. Insects in the order Orthoptera include but are not limited Melanoplus spp. such as M. diferentialis (Differential grasshopper), M. femurrubrum (Redlegged grasshopper), M. bivittatus (Twostriped grasshopper); and any combination thereof. Insects in the order Thysanoptera include but are not limited Frankliniella spp. such as F. occidentalis (western flower thrips) and F. fusca (tobacco thrips); and Thrips spp. such as T. tabaci (onion thrips), T. palmi (melon thrips); and any combination of the foregoing.

The disclosed insecticidal protein(s) may also have insecticidal activity against any one or more of the following: Phyllophaga spp., Rhopalosiphum maidis, Pratylenchus penetrans, Melanotus cribulosus, Cyclocephala lurida, Limonius calfornicus, Tetranychus urticae, Haplothrips aculeatus, Tetranychus truncates, Anomala corpulenta, Oedaleus infernalis, Frankliniella tenuicornis, Tetranychus cinnabarinus, Aiolopus thalassinus tamulus, Trachea tokionis, Laodelphax striatellus, Holotrichia oblita, Dichelops furcatus, Diloboderus abderu, Dalbulus maidis, Astylus variegathus, Scaptocoris castanea, Locusta migratoria manilensis, Agriotes lineatus, Peregrinus maidis, Oscinella frit, Frankliniella williamsi, Zyginidia manaliensis, Atherigona soccata, Nicentrites testaceipes, Myllocerus undecimpustulatus, Atherigona naquii, Amsecta albistriga, Plodia interpuctella, Melanotus caudex, Microtermes spp., Atherigona oryzae, Tanymecus dilaticollis, Delphacodes kuschelli, Lepidiota stigma, Phyllophaga hellery, Tribolium castaneum, Pelopidas mathias, Oxya chinensis (Thunberg), Stenocranus pacificus, Scutigerella immaculata, Chrysodeixis chalcites, Euproctis sp. (Lymantriidae), Phyllotreata spp. (undulata), Reptalus panzer, Cyrtacanthacris tartarica Linnaeus, Orgyfa postica, Dactylispa lameyi, Patanga succincta Johanson, Tetranychus spp., Calomycterus sp., Adoretus compressus Weber, and Paratetranychus stickney.

In some aspects, the disclosure provides vectors that comprise the nucleic acid molecules of the disclosure. Examples of a vector include a plasmid, cosmid, phagemid, artificial chromosome, phage or viral vector. In embodiments, the vector is plant vector, e.g., for use in transformation of plants. In embodiments, the vector is a bacterial vector, e.g., for use in transformation of bacteria. Suitable vectors for plants, bacteria and other organisms are known in the art.

In some embodiments, a nucleic acid molecule or vector of the disclosure can also include sequences that encode other desired traits in addition to the insecticidal protein(s). Such expression cassettes comprising the stacked traits may be used to create plants, plant parts or plant cells having a desired phenotype with the stacked traits (i.e., molecular stacking). Such stacked combinations in plants can also be created by other methods including, but not limited to, cross breeding plants by any conventional methodology. If stacked by genetically transforming the plants, the nucleotide sequences of interest can be combined at any time and in any order. For example, a transgenic plant comprising one or more desired traits can be used as the target to introduce further traits by subsequent transformation. The additional nucleotide sequences can be introduced simultaneously in a co-transformation protocol with a nucleic acid molecule or vector of this disclosure. For example, if two nucleotide sequences will be introduced, they can be incorporated in separate cassettes (trans) or can be incorporated on the same cassette (cis). Expression of polynucleotides can be driven by the same promoter or by different promoters. It is further recognized that polynucleotides can be stacked at a desired genomic location using a site-specific nuclease or recombination system (e.g., FRT/Flp, Cre/Lox, TALE-endonucleases, zinc finger nucleases, CRISPR/Cas and related technologies). See U.S. Pat. Nos. 7,214,536, 8,921,332, 8,765,448, 5,527,695, 5,744,336, 5,910,415, 6,110,736, 6,175,058, 6,720,475, 6,455,315, 6,458,594 and US Patent Publication Nos. US2019093090, US2019264218, US2018327785, US2017240911, US2016208272, US2019062765.

In some embodiments, a nucleic acid molecule or vector of the disclosure can include an additional coding sequence for one or more polypeptides or double stranded RNA molecules (dsRNA) of interest for agronomic traits that primarily are of benefit to a seed company, grower or grain processor. A polypeptide of interest can be any polypeptide encoded by a nucleotide sequence of interest. Non-limiting examples of polypeptides of interest that are suitable for production in plants include those resulting in agronomically important traits such as herbicide resistance (also sometimes referred to as “herbicide tolerance”), virus resistance, bacterial pathogen resistance, insect resistance, nematode resistance, or fungal resistance. See, e.g., U.S. Pat. Nos. 5,569,823; 5,304,730; 5,495,071; 6,329,504; and 6,337,431. The polypeptide also can be one that increases plant vigor or yield (including traits that allow a plant to grow at different temperatures, soil conditions and levels of sunlight and precipitation), or one that allows identification of a plant exhibiting a trait of interest (e.g., a selectable marker, seed coat color, etc.). Various polypeptides of interest, as well as methods for introducing these polypeptides into a plant, are described, for example, in U.S. Pat. Nos. 4,761,373; 4,769,061; 4,810,648; 4,940,835; 4,975,374; 5,013,659; 5,162,602; 5,276,268; 5,304,730; 5,495,071; 5,554,798; 5,561,236; 5,569,823; 5,767,366; 5,879,903, 5,928,937; 6,084,155; 6,329,504 and 6,337,431; as well as US Patent Publication No. 2001/0016956.

Polynucleotides conferring resistance/tolerance to an herbicide that inhibits the growing point or meristem, such as an imidazalinone or a sulfonylurea can also be suitable in some embodiments. Exemplary polynucleotides in this category code for mutant ALS and AHAS enzymes as described, e.g., in U.S. Pat. Nos. 5,767,366 and 5,928,937. U.S. Pat. Nos. 4,761,373 and 5,013,659 are directed to plants resistant to various imidazalinone or sulfonamide herbicides. U.S. Pat. No. 4,975,374 relates to plant cells and plants containing a nucleic acid encoding a mutant glutamine synthetase (GS) resistant to inhibition by herbicides that are known to inhibit GS, e.g., phosphinothricin and methionine sulfoximine. U.S. Pat. No. 5,162,602 discloses plants resistant to inhibition by cyclohexanedione and aryloxyphenoxypropanoic acid herbicides. The resistance is conferred by an altered acetyl coenzyme A carboxylase (ACCase).

Polypeptides encoded by nucleotides sequences conferring resistance to glyphosate are also suitable for the disclosure. See, e.g., U.S. Pat. Nos. 4,940,835 and 4,769,061. U.S. Pat. No. 5,554,798 discloses transgenic glyphosate resistant maize plants, which resistance is conferred by an altered 5-enolpyruvyl-3-phosphoshikimate (EPSP) synthase gene.

Polynucleotides coding for resistance to phosphono compounds such as glufosinate ammonium or phosphinothricin, and pyridinoxy or phenoxy propionic acids and cyclohexones are also suitable. See, European Patent Application No. 0 242 246. See also, U.S. Pat. Nos. 5,879,903, 5,276,268 and 5,561,236.

Other suitable polynucleotides include those coding for resistance to herbicides that inhibit photosynthesis, such as a triazine and a benzonitrile (nitrilase) See, U.S. Pat. No. 4,810,648. Additional suitable polynucleotides coding for herbicide resistance include those coding for resistance to 2,2-dichloropropionic acid, sethoxydim, haloxyfop, imidazolinone herbicides, sulfonylurea herbicides, triazolopyrimidine herbicides, s-triazine herbicides and bromoxynil. Also suitable are polynucleotides conferring resistance to a protox enzyme, or that provide enhanced resistance to plant diseases; enhanced tolerance of adverse environmental conditions (abiotic stresses) including but not limited to drought, excessive cold, excessive heat, or excessive soil salinity or extreme acidity or alkalinity; and alterations in plant architecture or development, including changes in developmental timing. See, e.g., U.S. Patent Publication No. 2001/0016956 and U.S. Pat. No. 6,084,155.

Additional suitable polynucleotides include those coding for insecticidal polypeptides. These polypeptides may be produced in amounts sufficient to control, for example, insect pests (i.e., insect controlling amounts). It is recognized that the amount of production of an insecticidal polypeptide in a plant necessary to control insects or other pests may vary depending upon the cultivar, type of pest, environmental factors and the like. Polynucleotides useful for additional insect or pest resistance include, for example, those that encode toxins identified in Bacillus organisms. Polynucleotides comprising nucleotide sequences encoding Bacillus thuringiensis (Bt) Cry proteins from several subspecies have been cloned and recombinant clones have been found to be toxic to lepidopteran, dipteran and/or coleopteran insect larvae. Examples of such Bt insecticidal proteins include the Cry proteins such as Cry1Aa, Cry1Ab, Cry1Ac, Cry1B, Cry1C, Cry1D, Cry1Ea, Cry1Fa, Cry3A, Cry9A, Cry9B, Cry9C, and the like, as well as vegetative insecticidal proteins such as Vip1, Vip2, Vip3, and the like. A full list of Bt-derived proteins can be found on the worldwide web at Bacillus thuringiensis Toxin Nomenclature Database maintained by the University of Sussex (see also, Crickmore et al. (1998) Microbiol. Mol. Biol. Rev. 62:807-813).

In embodiments, an additional polypeptide is an insecticidal polypeptide derived from a non-Bt source, including without limitation, an alpha-amylase, a peroxidase, a cholesterol oxidase, a patatin, a protease, a protease inhibitor, a urease, an alpha-amylase inhibitor, a pore-forming protein, a chitinase, a lectin, an engineered antibody or antibody fragment, a Bacillus cereus insecticidal protein, a Xenorhabdus spp. (such as X. nematophila or X. bovienii) insecticidal protein, a Photorhabdus spp. (such as P. luminescens or P. asymobiotica) insecticidal protein, a Brevibacillus spp. (such as B. laterosporous) insecticidal protein, a Lysinibacillus spp. (such as L. sphearicus) insecticidal protein, a Chromobacterium spp. (such as C. subtsugae or C. piscinae) insecticidal protein, a Yersinia spp. (such as Y. entomophaga) insecticidal protein, a Paenibacillus spp. (such as P. propylaea) insecticidal protein, a Clostridium spp. (such as C. bifermentans) insecticidal protein, a Pseudomonas spp. (such as P. fluorescens) and a lignin.

Polypeptides that are suitable for production in plants further include those that improve or otherwise facilitate the conversion of harvested plants or plant parts into a commercially useful product, including, for example, increased or altered carbohydrate content or distribution, improved fermentation properties, increased oil content, increased protein content, improved digestibility, and increased nutraceutical content, e.g., increased phytosterol content, increased tocopherol content, increased stanol content or increased vitamin content. Polypeptides of interest also include, for example, those resulting in or contributing to a reduced content of an unwanted component in a harvested crop, e.g., phytic acid, or sugar degrading enzymes. By “resulting in” or “contributing to” is intended that the polypeptide of interest can directly or indirectly contribute to the existence of a trait of interest (e.g., increasing cellulose degradation by the use of a heterologous cellulase enzyme).

In some embodiments, the polypeptide contributes to improved digestibility for food or feed. Xylanases are hemicellulolytic enzymes that improve the breakdown of plant cell walls, which leads to better utilization of the plant nutrients by an animal. This leads to improved growth rate and feed conversion. Also, the viscosity of the feeds containing xylan can be reduced. Heterologous production of xylanases in plant cells also can facilitate lignocellulosic conversion to fermentable sugars in industrial processing.

Numerous xylanases from fungal and bacterial microorganisms have been identified and characterized (see, e.g., U.S. Pat. No. 5,437,992; Coughlin et al. (1993) “Proceedings of the Second TRICEL Symposium on Trichoderma reesei Cellulases and Other Hydrolases” Espoo; Souminen and Reinikainen, eds. (1993) Foundation for Biotechnical and Industrial Fermentation Research 8:125-135; U.S. Patent Publication No. 2005/0208178; and PCT Publication No. WO 03/16654). In particular, three specific xylanases (XYL-I, XYL-II, and XYL-III) have been identified in T. reesei (Tenkanen et al. (1992) Enzyme Microb. Technol. 14:566; Torronen et al. (1992) Bio/Technology 10:1461; and Xu et al. (1998) Appl. Microbiol. Biotechnol. 49:718).

In other embodiments, a polypeptide useful for the disclosure can be a polysaccharide degrading enzyme. Plants of this disclosure producing such an enzyme may be useful for generating, for example, fermentation feedstocks for bioprocessing. In some embodiments, enzymes useful for a fermentation process include alpha amylases, proteases, pullulanases, isoamylases, cellulases, hemicellulases, xylanases, cyclodextrin glycotransferases, lipases, phytases, laccases, oxidases, esterases, cutinases, granular starch hydrolyzing enzyme and other glucoamylases.

Polysaccharide-degrading enzymes include: starch degrading enzymes such as α-amylases (EC 3.2.1.1), glucuronidases (E.C. 3.2.1.131); exo-1,4-α-D glucanases such as amyloglucosidases and glucoamylase (EC 3.2.1.3), β-amylases (EC 3.2.1.2), α-glucosidases (EC 3.2.1.20), and other exo-amylases; starch debranching enzymes, such as a) isoamylase (EC 3.2.1.68), pullulanase (EC 3.2.1.41), and the like; b) cellulases such as exo-1,4-3-cellobiohydrolase (EC 3.2.1.91), exo-1,3-β-D-glucanase (EC 3.2.1.39), β-glucosidase (EC 3.2.1.21); c) L-arabinases, such as endo-1,5-α-L-arabinase (EC 3.2.1.99), α-arabinosidases (EC 3.2.1.55) and the like; d) galactanases such as endo-1,4-β-D-galactanase (EC 3.2.1.89), endo-1,3-β-D-galactanase (EC 3.2.1.90), α-galactosidase (EC 3.2.1.22), β-galactosidase (EC 3.2.1.23) and the like; e) mannanases, such as endo-1,4-β-D-mannanase (EC 3.2.1.78), β-mannosidase (EC 3.2.1.25), α-mannosidase (EC 3.2.1.24) and the like; f) xylanases, such as endo-1,4-β-xylanase (EC 3.2.1.8), β-D-xylosidase (EC 3.2.1.37), 1,3-β-D-xylanase, and the like; and g) other enzymes such as α-L-fucosidase (EC 3.2.1.51), α-L-rhamnosidase (EC 3.2.1.40), levanase (EC 3.2.1.65), inulanase (EC 3.2.1.7), and the like. In one embodiment, the α-amylase is the synthetic α-amylase, Amy797E, described is U.S. Pat. No. 8,093,453, herein incorporated by reference in its entirety.

Further enzymes which may be used with the disclosure include proteases, such as fungal and bacterial proteases. Fungal proteases include, but are not limited to, those obtained from Aspergillus. Trichoderma. Mucor and Rhizopus, such as A. niger. A. awamori. A. orywae and M. miehei. In some embodiments, the polypeptides of this disclosure can be cellobiohydrolase (CBH) enzymes (EC 3.2.1.91). In one embodiment, the cellobiohydrolase enzyme can be CBH1 or CBH2.

Other enzymes useful with the disclosure include, but are not limited to, hemicellulases, such as mannases and arabinofuranosidases (EC 3.2.1.55); ligninases; lipases (e.g., E.C. 3.1.1.3), glucose oxidases, pectinases, xylanases, transglucosidases, alpha 1,6 glucosidases (e.g., E.C. 3.2.1.20); esterases such as ferulic acid esterase (EC 3.1.1.73) and acetyl xylan esterases (EC 3.1.1.72); and cutinases (e.g. E.C. 3.1.1.74).

Double stranded RNA molecules useful with the disclosure include but are not limited to those that suppress target insect genes. As used herein the words “gene suppression”, when taken together, are intended to refer to any of the well-known methods for reducing the levels of protein produced as a result of gene transcription to mRNA and subsequent translation of the mRNA. Gene suppression is also intended to mean the reduction of protein expression from a gene or a coding sequence including posttranscriptional gene suppression and transcriptional suppression. Posttranscriptional gene suppression is mediated by the homology between of all or a part of a mRNA transcribed from a gene or coding sequence targeted for suppression and the corresponding double stranded RNA used for suppression, and refers to the substantial and measurable reduction of the amount of available mRNA available in the cell for binding by ribosomes. The transcribed RNA can be in the sense orientation to effect what is called co-suppression, in the anti-sense orientation to effect what is called anti-sense suppression, or in both orientations producing a dsRNA to effect what is called RNA interference (RNAi). Transcriptional suppression is mediated by the presence in the cell of a dsRNA, a gene suppression agent, exhibiting substantial sequence identity to a promoter DNA sequence or the complement thereof to effect what is referred to as promoter trans suppression. Gene suppression may be effective against a native plant gene associated with a trait, e.g., to provide plants with reduced levels of a protein encoded by the native gene or with enhanced or reduced levels of an affected metabolite. Gene suppression can also be effective against target genes in plant pests that may ingest or contact plant material containing gene suppression agents, specifically designed to inhibit or suppress the expression of one or more homologous or complementary sequences in the cells of the pest. Such genes targeted for suppression can encode an essential protein, the predicted function of which is selected from the group consisting of muscle formation, juvenile hormone formation, juvenile hormone regulation, ion regulation and transport, digestive enzyme synthesis, maintenance of cell membrane potential, amino acid biosynthesis, amino acid degradation, sperm formation, pheromone synthesis, pheromone sensing, antennae formation, wing formation, leg formation, development and differentiation, egg formation, larval maturation, digestive enzyme formation, hemolymph synthesis, hemolymph maintenance, neurotransmission, cell division, energy metabolism, respiration, and apoptosis.

### Transgenic Cells, Plants, Plant Parts

In some aspects, the disclosure further provides transgenic cells, plants, plant parts, and the like comprising a nucleic acid molecule or vector of the disclosure (e.g., comprising any one of SEQ ID NOs: 1 or 8 to 31 or any one or more of the variants in Table 3). In some embodiments, the disclosure provides a non-human host cell comprising a nucleic acid molecule or vector of the disclosure. The transgenic non-human host cell can include, but is not limited to, a plant cell (including a monocot cell and/or a dicot cell), a yeast cell, a bacterial cell or an insect cell. Accordingly, in some embodiments, a bacterial cell is provided which is selected from the genera Bacillus, Brevibacillus, Clostridium, Xenorhabdus, Photorhabdus, Pasteuria, Escherichia, Pseudomonas, Erwinia, Serratia, Klebsiella, Salmonella, Pasteurella, Xanthomonas, Streptomyces, Rhizobium, Rhodopseudomonas, Methylophilius, Agrobacterium, Acetobacter, Lactobacillus, Arthrobacter, Azotobacter, Leuconostoc, or Alcaligenes.

In some embodiments, the transgenic plant cell is a dicot plant cell or a monocot plant cell. In additional embodiments, the dicot plant cell is a soybean cell, sunflower cell, tomato cell, cole crop cell, cotton cell, sugar beet cell or a tobacco cell. In further embodiments, the monocot cell is a barley cell, maize cell, oat cell, rice cell, sorghum cell, sugar cane cell or wheat cell. In preferred embodiments, the monocot cell is a maize cell. In some embodiments, the disclosure provides a plurality of dicot cells or monocot cells comprising a nucleic acid molecule or vector of the disclosure (e.g., a plurality of maize cells comprising a nucleic acid molecule or vector of the disclosure). In embodiments, the plurality of cells is juxtaposed to form an apoplast and are grown in natural sunlight. In embodiments, the transgenic plant cell cannot regenerate a whole plant.

In other embodiments of the disclosure, the nucleic acid molecule of the disclosure is expressed in a higher organism, for example, a plant. Such transgenic plants express effective amounts of the insecticidal protein(s) encoded by the nucleic acid molecule to control plant pests such as insect pests. When an insect starts feeding on such a transgenic plant, it ingests the expressed insecticidal protein(s). This can deter the insect from further biting into the plant tissue or may even harm or kill the insect. In some embodiments, the nucleic acid molecule of the disclosure is stably integrated in the genome of the plant. In other embodiments, the nucleic acid molecule of the disclosure is included in a non-pathogenic self-replicating virus.

In some embodiments, the transgenic plant is insecticidal against at least Spodoptera frugiperda (fall armyworm). In some embodiments, the transgenic plant is insecticidal against at least two (e.g., 2, 3, or 4) of Spodoptera frugiperda (fall armyworm), Mythimna separata (oriental armyworm), Spodoptera litura (common cutworm/oriental leafworm), and Ostrinia furnacalis (Asian corn borer). In some embodiments, the transgenic plant has enhanced insecticidal properties, e.g., against at least Spodoptera frugiperda (fall armyworm), relative to a control plant, e.g., that does not comprise the nucleic acid molecule.

In some embodiments of the disclosure, a transgenic plant cell comprising a nucleic acid molecule of the disclosure is a cell of a plant part, a plant organ or a plant culture (each as described herein) including, but not limited to, a root, a leaf, a seed, a flower, a fruit, a pollen cell, organ or plant culture, and the like, or a callus cell or culture.

A transgenic plant or plant cell transformed in accordance with the disclosure may be a monocot or dicot plant or plant cell and includes, but is not limited to, corn (maize), soybean, rice, wheat, barley, rye, oats, sorghum, millet, sunflower, safflower, sugar beet, cotton, sugarcane, oilseed rape, alfalfa, tobacco, peanuts, vegetables, including, sweet potato, bean, pea, chicory, lettuce, cabbage, cauliflower, broccoli, turnip, carrot, eggplant, cucumber, radish, spinach, potato, tomato, asparagus, onion, garlic, melons, pepper, celery, squash, pumpkin, zucchini, fruits, including, apple, pear, quince, plum, cherry, peach, nectarine, apricot, strawberry, grape, raspberry, blackberry, pineapple, avocado, papaya, mango, banana, and specialty plants, such as Arabidopsis, and woody plants such as coniferous and deciduous trees. Preferably, plants of the of the disclosure are crop plants such as maize, sorghum, wheat, sunflower, tomato, crucifers, peppers, potato, cotton, rice, soybean, sugar beet, sugarcane, tobacco, barley, oilseed rape, and the like.

Once a desired nucleic acid molecule has been transformed into a particular plant species, it may be propagated in that species or moved into other varieties of the same species, particularly including commercial varieties, using any appropriate technique including traditional breeding techniques.

The insecticidal protein(s) encoded by a nucleic acid molecule of the disclosure can function in the plant part, plant cell, plant organ, seed, harvested product, processed product or extract, and the like, as an insect control agent. In other words, the insecticidal protein(s) can continue to perform the insecticidal function it had in the transgenic plant. The nucleic acid molecule can function to express the insecticidal protein. As an alternative to expressing the insecticidal protein of the disclosure, in some embodiments the nucleic acid molecule can function to identify a transgenic plant part, plant cell, plant organ, seed, harvested product, processed product or extract of the disclosure comprising the nucleic acid molecule.

In embodiments, a transgenic plant, plant part, plant cell, plant organ, or seed of the disclosure is hemizygous for a nucleic acid molecule of the disclosure. In embodiments, a transgenic plant, plant part, plant cell, plant organ, or seed of the disclosure is homozygous for a nucleic acid molecule of the disclosure.

Additional embodiments of the disclosure include harvested products produced from the transgenic plants or parts thereof of the disclosure, as well as a processed product produced from the harvested products. A harvested product can be a whole plant or any plant part, as described herein. Thus, in some embodiments, non-limiting examples of a harvested product include a seed, a fruit, a flower or part thereof (e.g., an anther, a stigma, and the like), a leaf, a stem, and the like. In other embodiments, a processed product includes, but is not limited to, a flour, meal, oil, starch, syrup, cereal, and the like produced from a harvested seed or other plant part of the disclosure, wherein said seed or other plant part comprises a nucleic acid molecule of the disclosure.

In other embodiments, the disclosure provides an extract from a transgenic seed or a transgenic plant of the disclosure, wherein the extract comprises a nucleic acid molecule of the disclosure. Extracts from plants or plant parts can be made according to procedures well known in the art (See, de la Torre et al., Food, Agric. Environ. 2(1):84-89 (2004); Guidet, Nucleic Acids Res. 22(9): 1772-1773 (1994); Lipton et al., Food Agric. Immun. 12:153-164 (2000)). Such extracts may be used, e.g., in methods to detect the presence of a nucleic acid molecule of the disclosure.

In some embodiments, a transgenic plant, plant part, plant cell, plant organ, seed, harvested product, processed product or extract has increased insecticidal activity to one or more insect pests (e.g., a lepidopteran pest) as compared with a suitable control that does not comprise a nucleic acid molecule of the disclosure. In some embodiments, a transgenic plant, plant part, plant cell, plant organ, seed, harvested product, processed product or extract has increased insecticidal activity to at least Spodoptera frugiperda (fall armyworm). In some embodiments, a transgenic plant, plant part, plant cell, plant organ, seed, harvested product, processed product or extract has increased insecticidal activity to at least two (e.g., 2, 3, or 4) of Spodoptera frugiperda (fall armyworm), Mythimna separata (oriental armyworm), Spodoptera litura (common cutworm/oriental leafworm), and Ostrinia furnacalis (Asian corn borer).

### Plant Transformation and Breeding

Procedures for transforming plants are well known and routine in the art and are described throughout the literature. Non-limiting examples of methods for transformation of plants include transformation via bacterial-mediated nucleic acid delivery (e.g., via Agrobacterium), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) or biological mechanism that results in the introduction of a nucleic acid molecule into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

For Agrobacterium-mediated transformation, binary vectors or vectors carrying at least one T-DNA border sequence are generally suitable, whereas for direct gene transfer (e.g., particle bombardment and the like) any vector is suitable and linear DNA containing only the construction of interest can be used. In the case of direct gene transfer, transformation with a single DNA species or co-transformation can be used (Schocher et al., Biotechnology 4:1093-1096 (1986)). For both direct gene transfer and Agrobacterium-mediated transfer, transformation is usually (but not necessarily) undertaken with a selectable marker that may be a positive selection (e.g., Phosphomannose Isomerase), provide resistance to an antibiotic (e.g., kanamycin, hygromycin or methotrexate) or a herbicide (e.g., glyphosate or glufosinate). However, the choice of selectable marker is not critical to the disclosure.

Agrobacterium-mediated transformation is a commonly used method for transforming plants because of its high efficiency of transformation and because of its broad utility with many different species. Agrobacterium-mediated transformation typically involves transfer of the binary vector carrying the foreign DNA of interest to an appropriate Agrobacterium strain that may depend on the complement of vir genes carried by the host Agrobacterium strain either on a co-resident Ti plasmid or chromosomally (Uknes et al. (1993) Plant Cell 5:159-169). The transfer of the recombinant binary vector to Agrobacterium can be accomplished by a triparental mating procedure using Escherichia coli carrying the recombinant binary vector, a helper E. coli strain that carries a plasmid that is able to mobilize the recombinant binary vector to the target Agrobacterium strain. Alternatively, the recombinant binary vector can be transferred to Agrobacterium by nucleic acid transformation (Hö\ddot{f}gen & Willmitzer (1988) Nucleic Acids Res. 16:9877).

Dicots as well as monocots may be transformed using Agrobacterium. Methods for Agrobacterium-mediated transformation of rice include well known methods for rice transformation, such as those described in any of the following: European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of maize transformation, methods include those as described in either Ishida et al. (Nat. Biotechnol 14(6): 745-50, 1996) or Frame et al. (Plant Physiol 129(1): 13-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acid or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants, such as plants used as a model, like Arabidopsis or crop plants such as, by way of example, tobacco plants, for example by immersing bruised leaves or chopped leaves in an Agrobacterial solution and then culturing them in suitable media. The transformation of plants by means of Agrobacterium tumefaciens is described, for example, by Hagen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

Soybean plant material can be suitably transformed, and fertile plants regenerated by many methods which are well known to one of skill in the art. Examples of soybean transformation methods can be found in U.S. Pat. No. 5,024,944; Finer and McMullen (1991) In Vitro Cell Dev. Biol. 27P:175-182; McCabe et al. (1988) Bio/technology 6:923-926; Khalafalla et al. (2006) African J. of Biotechnology 5:1594-1599; U.S. Pat. No. 7,001,754; Hinchee et al. (1988) Bio/Technology 6:915-922; U.S. Pat. No. 7,002,058; U.S. Patent Application Publication No. 20040034889; U.S. Patent Application Publication No. 20080229447; and Paz et al. (2006) Plant Cell Report 25:206-213.

Transgenic plants can be generated with the heretofore described binary vectors containing selectable marker genes with different transformation methods. For example, a vector is used to transform immature seed targets as described (see e.g., U.S. Patent Application Publication No. 20080229447) to generate transgenic HPPD plants directly using HPPD inhibitor, such as mesotrione, as selection agent. Optionally, other herbicide tolerance genes can be present in the polynucleotide alongside other sequences which provide additional means of selection/identification of transformed tissue including, for example, the known genes which provide resistance to kanamycin, hygromycin, phosphinothricin, butafenacil, or glyphosate. For example, different binary vectors containing PAT or EPSPS selectable marker genes are transformed using Agrobacterium-mediated transformation and glufosinate or glyphosate selection as described (see e.g., U.S. Patent Application Publication No. 20080229447).

Transformation of a plant by recombinant Agrobacterium usually involves co-cultivation of the Agrobacterium with explants from the plant and follows methods well known in the art. Transformed tissue is regenerated on selection medium carrying an antibiotic or herbicide resistance marker between the binary plasmid T-DNA borders.

As discussed previously, another method for transforming plants, plant parts and plant cells involves propelling inert or biologically active particles at plant tissues and cells. See, e.g., U.S. Pat. Nos. 4,945,050; 5,036,006 and 5,100,792. Generally, this method involves propelling inert or biologically active particles at the plant cells under conditions effective to penetrate the outer surface of the cell and afford incorporation within the interior thereof. When inert particles are utilized, the vector can be introduced into the cell by coating the particles with the vector containing the nucleic acid of interest. Alternatively, a cell or cells can be surrounded by the vector so that the vector is carried into the cell by the wake of the particle. Biologically active particles (e.g., a dried yeast cell, a dried bacterium or a bacteriophage, each containing one or more nucleic acids sought to be introduced) also can be propelled into plant tissue.

In other embodiments, a nucleic acid molecule of the disclosure can be directly transformed into the plastid genome. Plastid transformation technology is extensively described in U.S. Pat. Nos. 5,451,513, 5,545,817, and 5,545,818, in PCT application no. WO 95/16783, and in McBride et al. (1994) Proc. Natl. Acad. Sci. USA 91, 7301-7305.

Methods of selecting for transformed, transgenic plants, plant cells or plant tissue culture are routine in the art and can be employed in the methods of the disclosure provided herein. For example, a nucleic acid molecule or vector of the disclosure also can include an expression cassette comprising a nucleotide sequence for a selectable marker, which can be used to select a transformed plant, plant part or plant cell.

Examples of selectable markers include, but are not limited to, a nucleotide sequence encoding neo or nptII, which confers resistance to kanamycin, G418, and the like (Potrykus et al. (1985) Mol. Gen. Genet. 199:183-188); a nucleotide sequence encoding bar, which confers resistance to phosphinothricin; a nucleotide sequence encoding an altered 5-enolpyruvylshikimate-3-phosphate (EPSP) synthase, which confers resistance to glyphosate (Hinchee et al. (1988) Biotech. 6:915-922); a nucleotide sequence encoding a nitrilase such as bxn from Klebsiella ozaenae that confers resistance to bromoxynil (Stalker et al. (1988) Science 242:419-423); a nucleotide sequence encoding an altered acetolactate synthase (ALS) that confers resistance to imidazolinone, sulfonylurea or other ALS-inhibiting chemicals (EP Patent Application No. 154204); a nucleotide sequence encoding a methotrexate-resistant dihydrofolate reductase (DHFR) (Thillet et al. (1988) J. Biol. Chem. 263:12500-12508); a nucleotide sequence encoding a dalapon dehalogenase that confers resistance to dalapon; a nucleotide sequence encoding a mannose-6-phosphate isomerase (also referred to as phosphomannose isomerase (PMI)) that confers an ability to metabolize mannose (U.S. Pat. Nos. 5,767,378 and 5,994,629); a nucleotide sequence encoding an altered anthranilate synthase that confers resistance to 5-methyl tryptophan; or a nucleotide sequence encoding hph that confers resistance to hygromycin. One of skill in the art is capable of choosing a suitable selectable marker for use in an expression cassette of this disclosure.

Additional selectable markers include, but are not limited to, a nucleotide sequence encoding β-glucuronidase or uidA (GUS) that encodes an enzyme for which various chromogenic substrates are known; an R-locus nucleotide sequence that encodes a product that regulates the production of anthocyanin pigments (red color) in plant tissues (Dellaporta et al., “Molecular cloning of the maize R-nj allele by transposon-tagging with Ac” 263-282 In: Chromosome Structure and Function: Impact of New Concepts, 18th Stadler Genetics Symposium (Gustafson & Appels eds., Plenum Press 1988)); a nucleotide sequence encoding β-lactamase, an enzyme for which various chromogenic substrates are known (e.g., PADAC, a chromogenic cephalosporin) (Sutcliffe (1978) Proc. Natl. Acad. Sci. USA 75:3737-3741); a nucleotide sequence encoding xylE that encodes a catechol dioxygenase (Zukowsky et al. (1983) Proc. Natl. Acad. Sci. USA 80:1101-1105); a nucleotide sequence encoding tyrosinase, an enzyme capable of oxidizing tyrosine to DOPA and dopaquinone, which in turn condenses to form melanin (Katz et al. (1983) J. Gen. Microbiol. 129:2703-2714); a nucleotide sequence encoding β-galactosidase, an enzyme for which there are chromogenic substrates; a nucleotide sequence encoding luciferase (lux) that allows for bioluminescence detection (Ow et al. (1986) Science 234:856-859); a nucleotide sequence encoding aequorin which may be employed in calcium-sensitive bioluminescence detection (Prasher et al. (1985) Biochem. Biophys. Res. Comm. 126:1259-1268); or a nucleotide sequence encoding green fluorescent protein (Niedz et al. (1995) Plant Cell Reports 14:403-406) or other fluorescent protein such as dsRed or mCherry. One of skill in the art is capable of choosing a suitable selectable marker for use in an expression cassette of this disclosure.

Further, as is well known in the art, intact transgenic plants can be regenerated from transformed plant cells, plant tissue culture or cultured protoplasts using any of a variety of known techniques. Plant regeneration from plant cells, plant tissue culture or cultured protoplasts is described, for example, in Evans et al. (Handbook of Plant Cell Cultures, Vol. 1, MacMilan Publishing Co. New York (1983)); and Vasil I. R. (ed.) (Cell Culture and Somatic Cell Genetics of Plants, Acad. Press, Orlando, Vol. I (1984), and Vol. II (1986)).

Additionally, the genetic properties engineered into the transgenic seeds and plants, plant parts, or plant cells of the disclosure described above can be passed on by sexual reproduction or vegetative growth and therefore can be maintained and propagated in progeny plants. Generally, maintenance and propagation make use of known agricultural methods developed to fit specific purposes such as harvesting, sowing or tilling.

A nucleic acid molecule of the disclosure therefore can be introduced into the plant, plant part or plant cell in any number of ways that are well known in the art, as described above. Therefore, no particular method for introducing a nucleic acid molecule into a plant is relied upon, rather any method that allows the nucleic acid molecule to be stably integrated into the genome of the plant can be used. Where more than one polynucleotide is to be introduced, the respective polynucleotides can be assembled as part of a single nucleic acid molecule, or as separate nucleic acid molecules, and can be located on the same or different nucleic acid molecules. Accordingly, the polynucleotides can be introduced into the cell of interest in a single transformation event, in separate transformation events, or, for example, in plants, as part of a breeding protocol.

Once a desired nucleic acid molecule has been transformed into a particular plant species, it may be propagated in that species or moved into other varieties of the same species, particularly including commercial varieties, using traditional breeding techniques.

In some embodiments, a transgenic plant, plant part, plant cell, plant organ, seed, harvested product, processed product or extract of the disclosure can comprise one or more other nucleic acids of interest that provide one or more input traits (e.g., insect resistance, herbicide resistance, fungal resistance, virus resistance, stress tolerance, disease resistance, male sterility, stalk strength, and the like) and/or output traits (e.g., increased yield, modified starches, improved oil profile, balanced amino acids, high lysine or methionine, increased digestibility, improved fiber quality, drought resistance, and the like). In some embodiments, a transgenic plant of the disclosure can bred be with another transgenic plant comprising one or more other nucleic acids of interest.

In some embodiments, one or more other nucleic acids of interest encode one or more second pest control agents, e.g., a Bacillus thuringiensis (Bt) insecticidal protein, and/or a non-Bt insecticidal agent including without limitation a Xenorhabdus insecticidal protein, a Photorhabdus insecticidal protein, a Brevibacillus laterosporus insecticidal protein, a Bacillus sphaericus insecticidal protein, a protease inhibitor (both serine and cysteine types), a lectin, an alpha-amylase, a peroxidase, a cholesterol oxidase, or a double stranded RNA (dsRNA) molecule. In additional embodiments, the second pest control agent can be one or more of any number of Bacillus thuringiensis insecticidal proteins including but not limited to a Cry protein, a vegetative insecticidal protein (VIP) and insecticidal chimeras of any of the preceding insecticidal proteins. In some embodiments, the second pest control agent can be non-proteinaceous, for example, an interfering RNA molecule such as a dsRNA.

In some embodiments, the second pest control agent comprises any one or more of the insecticidal proteins or dsRNAs present in any of the following events: the Bt11 event (see U.S. Pat. No. 6,114,608), the MIR604 event (see U.S. Pat. No. 8,884,102), the MIR162 event (see U.S. Pat. No. 8,232,456), the 5307 event (see U.S. Pat. No. 10,428,393), the MZIR098 event (see US Patent Application No. US20200190533), the TC1507 event (see U.S. Pat. No. 7,288,643), the DAS-59122-7 event (see U.S. Pat. No. 7,323,556), the MON810 event (see U.S. Pat. No. 6,713,259), the MON863 event (see U.S. Pat. No. 7,705,216), the MON89034 event (see U.S. Pat. No. 8,062,840), the MON88017 event (see U.S. Pat. No. 9,556,492), the DP-4114 event (see U.S. Pat. No. 9,725,772), the MON87411 event (see U.S. Pat. No. 9,441,240), the DP-032218-9 event (see US Patent Application No. US2015361447), the DP-033121-3 event (see US Patent Application No. US2015361446), the DP-023211-2 event (see PCT Publication No. WO2019209700), the MON95379 event (see US Patent Application No. US2020032289), the DBN9936 event (see PCT Publication No. WO2016173361), the DBN9501 event (see PCT Publication No. WO20207125), the GH5112E-117C event (see PCT Publication No. WO17/088480), LP007-1 (see Chinese Patent Application No. CN112852801), LP007-2 (Chinese Patent Application No. CN112831584), LP007-3 (Chinese Patent Application No. CN112877454), LP007-4 (Chinese Patent Application No. CN112831585), LP007-5 (Chinese Patent Application No. CN113151534), LP007-6 (Chinese Patent Application No. CN113151533), LP007-7 (Chinese Patent Application No. CN112852991), LP007-8 (CN113980958), Ruifeng8, ND207, or the Ruifeng125 event (see Chinese Patent Application No. CN105017391). In some embodiments, the second pest control agent comprises any one or more of the following events: the Bt11 event (see U.S. Pat. No. 6,114,608), the MIR604 event (see U.S. Pat. No. 8,884,102), the MIR162 event (see U.S. Pat. No. 8,232,456), the 5307 event (see U.S. Pat. No. 10,428,393), the MZIR098 event (see US Patent Application No. US20200190533), the TC1507 event (see U.S. Pat. No. 7,288,643), the DAS-59122-7 event (see U.S. Pat. No. 7,323,556), the MON810 event (see U.S. Pat. No. 6,713,259), the MON863 event (see U.S. Pat. No. 7,705,216), the MON89034 event (see U.S. Pat. No. 8,062,840), the MON88017 event (see U.S. Pat. No. 9,556,492), the DP-4114 event (see U.S. Pat. No. 9,725,772), the MON87411 event (see U.S. Pat. No. 9,441,240), the DP-032218-9 event (see US Patent Application No. US2015361447), the DP-033121-3 event (see US Patent Application No. US2015361446), the DP-023211-2 event (see PCT Publication No. WO2019209700), the MON95379 event (see US Patent Application No. US2020032289), the DBN9936 event (see PCT Publication No. WO2016173361), the DBN9501 event (see PCT Publication No. WO20207125), the GH5112E-117C event (see PCT Publication No. WO17/088480), LP007-1 (see Chinese Patent Application No. CN112852801), LP007-2 (Chinese Patent Application No. CN112831584), LP007-3 (Chinese Patent Application No. CN112877454), LP007-4 (Chinese Patent Application No. CN112831585), LP007-5 (Chinese Patent Application No. CN113151534), LP007-6 (Chinese Patent Application No. CN113151533), LP007-7 (Chinese Patent Application No. CN112852991), LP007-8 (CN113980958), Ruifeng8, ND207, or the Ruifeng125 event (see Chinese Patent Application No. CN105017391).

In embodiments, the second pest control agent may be derived from sources other than B. thuringiensis. For example, the second pest control agent can be an alpha-amylase, a peroxidase, a cholesterol oxidase, a patatin, a protease, a protease inhibitor, a urease, an alpha-amylase inhibitor, a pore-forming protein, a chitinase, a lectin, an engineered antibody or antibody fragment, a Bacillus cereus insecticidal protein, a Xenorhabdus spp. (such as X. nematophila or X. bovienii) insecticidal protein, a Photorhabdus spp. (such as P. luminescens or P. asymobiotica) insecticidal protein, a Brevibacillus spp. (such as B. laterosporous) insecticidal protein, a Lysinibacillus spp. (such as L. sphearicus) insecticidal protein, a Chromobacterium spp. (such as C. subtsugae or C. piscinae) insecticidal protein, a Yersinia spp. (such as Y. entomophaga) insecticidal protein, a Paenibacillus spp. (such as P. propylaea) insecticidal protein, a Clostridium spp. (such as C. bifermentans) insecticidal protein, a Pseudomonas spp. (such as P. fluorescens) and a lignin. In other embodiments, the second agent may be at least one insecticidal protein derived from an insecticidal toxin complex (Tc) from Photorhabdus, Xenorhabus, Serratia, or Yersinia. In other embodiments, the insecticidal protein may be an ADP-ribosyltransferase derived from an insecticidal bacteria, such as Photorhabdus ssp. In other embodiments, the insecticidal protein may be a VIP protein, such as VIP1 and/or VIP2 from B. cereus. In still other embodiments, the insecticidal protein may be a binary toxin derived from an insecticidal bacterium, such as ISP1A and ISP2A from B. laterosporous or BinA and BinB from L. sphaericus. In still other embodiments, the insecticidal protein may be engineered or may be a hybrid or chimera of any of the preceding insecticidal proteins.

In some embodiments, one or more other nucleic acids of interest encode one or more herbicide tolerance agents, e.g., PAT (phosphinothricin N-acetyltransferase), AAD-1 (aryloxyalkanoate dioxygenase 1), EPSPS (5-enolpyruvulshikimate-3-phosphate synthase), or inhibitors of protoporphyrinogen oxidase (PPO, see, e.g., US Patent Application No. US2019185873). In some embodiments, the herbicide tolerance agent comprises any one or more of the following events: GA21 (see PCT Publication No. WO98/44140), NK603 (see U.S. Pat. No. 6,825,400), DAS40278 (see PCT Publication No. WO2011/022469), DBN9858 (see PCT Publication No. WO2016173508), MON87429 (see PCT Publication No. WO19/152316), LW2-2 (see Chinese Patent Application No. CN113278721) and T25 (see USDA/APHIS Petition 94-357-01 for Determination of Nonregulated Status for Glufosinate Resistant Corn Transformation Events T14 and T25, June 1995).

In some embodiments, one or more other nucleic acids of interest encode one or more enzymes, e.g., an alpha-amylase. In some embodiments, the enzyme comprises the 3272 event (see U.S. Pat. No. 7,635,799).

In some embodiments, one or more other nucleic acids of interest comprise one or more of the following events: MZDT09Y (see U.S. Pat. No. 9,121,033), LY038 (see U.S. Pat. No. 7,157,281), BT176 (see Koziel et al. (1993) Biotechnology 11: 194-200), and DP202216-6 (see US Patent Application No US2019320607).

Transgenic plants or seed comprising a nucleic acid molecule of the disclosure can also be treated with an insecticide or insecticidal seed coating as described, e.g., in U.S. Pat. Nos. 5,849,320 and 5,876,739. In some embodiments, both the insecticide or insecticidal seed coating and the transgenic plant or seed of the disclosure are active against the same target insect, for example a lepidopteran pest (e.g., fall armyworm). Thus, in some embodiments, a method is provided of enhancing control of a lepidopteran insect population comprising providing a transgenic plant or seed of the disclosure and applying to the plant or the seed an insecticide or insecticidal seed coating.

Even where the insecticide or insecticidal seed coating is active against a different insect, the insecticide or insecticidal seed coating is useful to expand the range of insect control, for example by adding an insecticide or insecticidal seed coating that has activity against coleopteran insects to a transgenic seed of the disclosure, which, in some embodiments, has activity against lepidopteran insects, the coated transgenic seed produced controls both lepidopteran and coleopteran insect pests.

### Methods of Using Nucleic Acid Molecules and Transgenic Plants

In some aspects, the disclosure also provides methods of producing and using a nucleic acid molecule of the disclosure and related compositions such as cells and plants comprising the nucleic acid molecule and uses thereof.

In some embodiments, the methods of the disclosure provide control of at least one lepidopteran insect pest, including without limitation, one or more of the following: Spodoptera spp. such as S. frugiperda (fall armyworm), S. littoralis (Egyptian cotton leafworm), S. ornithogalli (yellowstriped armyworm), S. praefica (western yellowstriped armyworm), S. eridania (southern armyworm), S. litura (Common cutworm/Oriental leafworm), S. cosmioides (black armyworm), S. exempta (African armyworm), S. mauritia (lawn armyworm) and/or S. exigua (beet armyworm); Ostrinia spp. such as O. nubilalis (European corn borer) and/or O. furnacalis (Asian corn borer); Plutella spp. such as P. xylostella (diamondback moth); Agrotis spp. such as A. ipsilon (black cutworm), A. segetum (common cutworm), A. gladiaria (claybacked cutworm), and/or A. orthogonia (pale western cutworm); Striacosta spp. such as S. albicosta (western bean cutworm); Helicoverpa spp. such as H. zea (corn earworm/soybean podworm), H. punctigera (native budworm), and/or H. armigera (cotton bollworm); Heliothis spp. such as H. virescens (tobacco budworm); Diatraea spp. such as D. grandiosella (southwestern corn borer) and/or D. saccharalis (sugarcane borer); Trichoplusia spp. such as T. ni (cabbage looper); Sesamia spp. such as S. nonagroides (Mediterranean corn borer), S. inferens (Pink stem borer) and/or S. calamistis (pink stem borer); Pectinophora spp. such as P. gossypiella (pink bollworm); Cochylis spp. such as C. hospes (banded sunflower moth); Manduca spp. such as M. sexta (tobacco hornworm) and/or M. quinquemaculata (tomato hornworm); Elasmopalpus spp. such as E. lignosellus (lesser cornstalk borer); Pseudoplusia spp. such as P. includens (soybean looper); Anticarsia spp. such as A. gemmatalis (velvetbean caterpillar); Plathypena spp. such as P. scabra (green cloverworm); Pieris spp. such as P. brassicae (cabbage butterfly), Papaipema spp. such as P. nebris (stalk borer); Pseudaletia spp. such as P. unipuncta (common armyworm); Peridroma spp. such as P. saucia (variegated cutworm); Keiferia spp. such as K. lycopersicella (tomato pinworm); Artogeia spp. such as A. rapae (imported cabbageworm); Phthorimaea spp. such as P. operculella (potato tuberworm); Chrysodeixis spp. such as C. includens (soybean looper); Feltia spp. such as F. ducens (dingy cutworm); Chilo spp. such as C. suppressalis (striped stem borer), C. Agamemnon (oriental corn borer), and C. partellus (spotted stalk borer), Cnaphalocrocis spp. such as C. medinalis (rice leaffolder), Conogethes spp. such as C. punctiferalis (Yellow peach moth), Mythimna spp. such as M. separata (Oriental armyworm), Athetis spp. such as A. lepigone (Two-spotted armyworm), Busseola spp. such as B. fusca (maize stalk borer), Etiella spp. such as E. zinckenella (pulse pod borer), Leguminivora spp. such as L. glycinivorella (soybean pod borer), Matsumuraeses spp. such as M. phaseoli (adzuki pod worm), Omiodes spp. such as O. indicata (Soybean leaffolder/Bean-leaf webworm), Rachiplusia spp. such as R. nu (sunflower Looper), or any combination of the foregoing. In some embodiments, the lepidopteran pest is at least S. frugiperda (fall armyworm). In some embodiments, the lepidopteran pest is at least two (e.g., 2, 3, or 4) of Spodoptera frugiperda (fall armyworm), Mythimna separata (oriental armyworm), Spodoptera litura (common cutworm/oriental leafworm), and Ostrinia furnacalis (Asian corn borer).

In some embodiments, the methods provide control of a fall armyworm insect pest or colony that is resistant to another insecticidal protein such as a Vip3A protein (e.g., a Vip3Aa, including without limitation maize event MIR162), a Cry1F protein (e.g., Cry1Fa, including without limitation maize event TC1507 or DP-4114), a Cry1A protein (e.g., Cry1A.105, including without limitation maize event MON89034), and/or a Cry2 protein (e.g., Cry2Ab, including without limitation maize event MON89034).

In further embodiments, a method of controlling a lepidopteran pest is provided, the method comprising delivering to the pest an effective amount of a plant or plant part comprising a nucleic acid molecule of the disclosure. To be effective, the insecticidal protein(s) expressed by the nucleic acid molecule of the disclosure is/are orally ingested by the pest. In some embodiments, the insecticidal protein(s) are delivered to the pest in a transgenic plant, wherein the pest eats (ingests) one or more parts of the transgenic plant, thereby ingesting the insecticidal protein(s) that is/are expressed in the transgenic plant.

Also encompassed are methods of producing a transgenic plant with enhanced insecticidal properties. In representative embodiments, the method comprises: introducing into a plant a nucleic acid molecule of the disclosure, wherein the nucleotide acid molecule is expressed in the plant to produce insecticidal protein(s), thereby conferring to the plant enhanced insecticidal properties.

In some embodiments, the method of introducing the nucleic acid molecule of the disclosure into the plant comprises first transforming a plant cell with the nucleic acid molecule of the disclosure and regenerating a transgenic plant therefrom, where the transgenic plant comprises the nucleic acid molecule of the disclosure. In some embodiments, the method comprises introducing into a plant, tissue culture, or a plant cell the nucleic acid molecule of the disclosure to obtain a transformed plant, transformed tissue culture, or a transformed cell having enhanced insecticidal properties; and growing the transformed plant or regenerating a transformed plant from the transformed tissue culture or transformed plant cell, so a transgenic plant with enhanced insecticidal properties is produced.

Alternatively, or additionally, the introducing step can comprise crossing a first plant comprising the nucleic acid molecule of the disclosure with a second plant (e.g., a different plant from the first plant, for example, a plant that does not comprise the nucleic acid molecule of the disclosure) and, optionally, producing a progeny plant that comprises the nucleic acid molecule of the disclosure. Thus, a transgenic plant encompasses a plant that is the direct result of a transformation event and the progeny thereof (of any generation) that comprise the nucleic acid molecule of the disclosure.

The disclosure further provides a method of identifying a transgenic plant of the disclosure, the method comprising detecting the presence of a nucleic acid molecule of the disclosure in a plant (or a plant cell, plant part, and the like derived therefrom), and thereby identifying the plant as a transgenic plant of the disclosure based on the presence of the nucleic acid molecule of the disclosure.

Some embodiments further provide a method of producing a transgenic plant with increased resistance to at least one insect pest (e.g., a least one lepidopteran pest), the method comprising: planting a seed comprising a nucleic acid molecule of the disclosure or vector of the disclosure, and growing a transgenic plant from the seed, where the transgenic plant comprises the nucleic acid molecule of the disclosure.

The methods of producing a transgenic plant described herein optionally comprise a further step of harvesting a seed from the transgenic plant, where the seed comprises the nucleic acid molecule of the disclosure. Optionally, the seed produces a further transgenic plant that comprises the nucleic acid molecule of the disclosure.

The disclosure further provides plant parts, plant cells, plant organs, plant cultures, seed, plant extracts, harvested products and processed products of the transgenic plants produced by the methods of the disclosure.

As a further aspect, the disclosure also provides a method of producing seed, the method comprising: providing a transgenic plant that comprises a nucleic acid molecule of the disclosure, and harvesting a seed from the transgenic plant, wherein the seed comprises the nucleic acid molecule of the disclosure. Optionally, the seed produces a further transgenic plant that comprises the nucleic acid molecule of the disclosure. In representative embodiments, the step of providing the transgenic plant comprises planting a seed that produces the transgenic plant.

Further provided is a method of producing a hybrid plant seed, the method comprising: crossing a first inbred plant, which is a transgenic plant comprising a nucleic acid molecule of the disclosure of the disclosure with a different inbred plant (e.g., an inbred plant that does not comprise a nucleic acid molecule of the disclosure) and allowing hybrid seed to form. Optionally, the method further comprises harvesting a hybrid seed. In some embodiments, the hybrid seed comprises the nucleic acid molecule of the disclosure. In some embodiments, the hybrid seed produces a transgenic plant that comprises the nucleic acid molecule of the disclosure.

In some embodiments, the disclosure provides a method of producing a commodity plant product, the method comprising using a transgenic plant comprising a nucleic acid molecule of the disclosure to produce said commodity plant product therefrom. Example commodity plant products include grain, starch, seed oil, syrup, flour, meal, starch, cereal, protein and the like. Methods of producing such commodity plant products are well known in the art.

In some aspects, the disclosure provides a method of detecting the presence of a nucleic acid molecule in a sample, the method comprising: (a) contacting the sample with a pair of primers that, when used in a nucleic-acid amplification reaction with DNA comprising the nucleic acid molecule of any of the above-mentioned embodiments or any other embodiment described herein (e.g., comprising any one of SEQ ID NOs: 1 or 8 to 31 or any one or more of the variants in Table 3), produces an amplicon that is diagnostic for the nucleic acid molecule; (b) performing a nucleic acid amplification reaction, thereby producing the amplicon; and (c) detecting the amplicon. In some embodiments, the pair of primers is a first primer and a second primer wherein the first primer comprises at least 10 (e.g., at least 10, at least 15 or at least 20) contiguous nucleotides that are complementary to any one of SEQ ID NOs: 1 or 8 to 31 or any one or more of the variants in Table 3 and the second primer comprises at least 10 contiguous nucleotides that are complementary to the reverse complement of any one of SEQ ID NOs: 1 or 8 to 31 or any one or more of the variants in Table 3. In some embodiments, the first and second primer are between 10-50, 10-40, 10-30, or 10-20 nucleotides in length. In some embodiments, the sample is a sample obtained from a maize plant part or cell.

In some aspects, the disclosure provides a method of detecting the presence of a nucleic acid molecule in a sample, the method comprising: (a) contacting the sample with a probe that hybridizes under high stringency conditions with DNA comprising the nucleic acid molecule of any of the above-mentioned embodiments or any other embodiment described herein (e.g., comprising any one of SEQ ID NOs: 1 or 8 to 31 or any one or more of the variants in Table 3) and does not hybridize under high stringency conditions with DNA of a control maize plant not comprising the nucleic acid molecule; (b) subjecting the sample and probe to high stringency hybridization conditions; and (c) detecting hybridization of the probe to the nucleic acid molecule. In some embodiments, the probe comprises at least 10 (e.g., at least 10, at least 15 or at least 20) contiguous nucleotides that are complementary to any one of SEQ ID NOs: 1 or 8 to 31 or any one or more of the variants in Table 3 or the reverse complement thereof. In some embodiments, the probe is between 10-50, 10-40, 10-30, or 10-20 nucleotides in length. In some embodiments, the sample is a sample obtained from a maize plant part or cell.

In some aspects, the disclosure provides a pair of polynucleotide primers comprising a first polynucleotide primer and a second polynucleotide primer which function together in the presence of the nucleic acid molecule of any of the above-mentioned embodiments or any other embodiment described herein (e.g., comprising any one of SEQ ID NOs: 1 or 8 to 31 or any one or more of the variants in Table 3) in a sample to produce an amplicon diagnostic for the presence of the nucleic acid molecule in a sample. In some embodiments, the sample is a sample obtained from a maize plant part or cell. In some embodiments, the first polynucleotide primer comprises at least 10 contiguous nucleotides that are complementary to any one of SEQ ID NOs: 1 or 8 to 31 or any one or more of the variants in Table 3 and the second polynucleotide primer comprises at least 10 (e.g., at least 10, at least 15 or at least 20) contiguous nucleotides that are complementary to the reverse complement of any one of SEQ ID NOs: 1 or 8 to 31 or any one or more of the variants in Table 3. In some embodiments, the first and second primer are between 10-50, 10-40, 10-30, or 10-20 nucleotides in length.

In some aspects, the disclosure provides a kit for detecting the nucleic acid molecule of any of the above-mentioned embodiments or any other embodiment described herein (e.g., comprising any one of SEQ ID NOs: 1 or 8 to 31 or any one or more of the variants in Table 3), the kit comprising at least one nucleic acid molecule of sufficient length of contiguous nucleotides to function as a primer or probe in a nucleic acid detection method, and which upon amplification of or hybridization to a target nucleic acid sequence in a sample followed by detection of the amplicon or hybridization to the target sequence, are diagnostic for the presence of the nucleic acid molecule. In some embodiments, the at least one nucleic acid molecule comprises at least 10 (e.g., at least 10, at least 15 or at least 20) contiguous nucleotides that are complementary to any one of SEQ ID NOs: 1 or 8 to 31 or any one or more of the variants in Table 3. In some embodiments, the at least one nucleic acid molecule comprises a pair of primers, wherein the first polynucleotide primer comprises at least 10 (e.g., at least 10, at least 15 or at least 20) contiguous nucleotides that are complementary to any one of SEQ ID NOs: 1 or 8 to 31 or any one or more of the variants in Table 3 and the second polynucleotide primer comprises at least 10 (e.g., at least 10, at least 15 or at least 20) contiguous nucleotides that are complementary to the reverse complement of any one of SEQ ID NOs: 1 or 8 to 31 or any one or more of the variants in Table 3. In some embodiments, the first and second primer are between 10-50, 10-40, 10-30, or 10-20 nucleotides in length. In some embodiments, the at least one nucleic acid molecule comprises a probe that comprises at least 10 contiguous nucleotides that are complementary to any one of SEQ ID NOs: 1 or 8 to 31 or any one or more of the variants in Table 3 or the reverse complement thereof. In some embodiments, the probe is between 10-50, 10-40, 10-30, or 10-20 nucleotides in length. Kits of the disclosure may optionally also comprise reagents and/or instructions for performing the detection as described herein.

In some aspects, the disclosure provides methods of modifying a nucleic acid molecule of the disclosure, e.g., in a cell or plant. In some embodiments, the modification is a deletion, an insertion (e.g., of a heterologous nucleic acid sequence), a substitution, a duplication, or inversion, or a combination thereof. In some embodiments, the modification comprises deletion of a portion or all of a selectable marker coding sequence present in the nucleic acid molecule, e.g., a PMI or EPSPS coding sequence. In some embodiments, the modification is introduced using a nuclease, such as a CRISPR-Cas nuclease, a Zinc finger nuclease, a meganuclease, a TAL effector nuclease (TALEN), or a combination thereof.

In some embodiments, the modification is made in a host cell or plant of the disclosure, e.g., a maize cell or maize plant, to produce a modified transgenic cell or modified transgenic plant. In some embodiments, the modification is made by expressing the nuclease in the host cell or plant (e.g., by transforming the host cell or plant with an expression cassette encoding the nuclease or by crossing the plant with another plant containing such an expression cassette). In some embodiments, the modification is made by directly introducing the nuclease into the host cell or plant, e.g., using reagents that transfer the nuclease into the host cell or plant such as through physical methods such as biolistics/particle bombardment, protoplast transfection, nanoparticle-mediated delivery, aerosol bean injection, or whisker-mediated delivery. In some embodiments, the method further comprises producing a plant from the modified transgenic host cell to produce a modified transgenic plant. In some embodiments, the method further comprises selfing or crossing the modified transgenic plant with another plant for at least one generation (e.g., one, two, three, four or more generations), thereby producing a modified transgenic progeny plant. In some embodiments, the disclose provides such a modified transgenic cell, modified transgenic plant, or modified transgenic progeny plant, e.g., produced by a method herein.

In certain embodiments, the nucleic acid modification is affected by a (modified) zinc-finger nuclease (ZFN) system. The ZFN system uses artificial restriction enzymes generated by fusing a zinc finger DNA-binding domain to a DNA-cleavage domain that can be engineered to target desired DNA sequences. Non-limiting examples of methods of using ZFNs can be found for example in U.S. Pat. Nos. 6,534,261; 6,607,882; 6,746,838; 6,794,136; 6,824,978; 6,866,997; 6,933,113; and 6,979,539.

In certain embodiments, the nucleic acid modification is affected by a meganuclease, which are endodeoxyribonucleases characterized by a large recognition site (double-stranded DNA sequences of 12 to 40 base pairs). Non-limiting examples of methods of using meganucleases can be found in U.S. Pat. Nos. 8,163,514; 8,133,697; 8,021,867; 8,119,361; 8,119,381; 8,124,369; and 8,129,134

In certain embodiments, the nucleic acid modification is affected by a CRISPR/Cas complex or system. In certain embodiments, the CRISPR/Cas system or complex is a class 2 CRISPR/Cas system. In certain embodiments, said CRISPR/Cas system or complex is a type II, type V, or type VI CRISPR/Cas system or complex. The CRISPR/Cas system does not require the generation of customized proteins to target specific sequences but rather a Cas nuclease can be programmed by an RNA guide (gRNA) to recognize a specific nucleic acid target, in other words the Cas nuclease can be recruited to a specific nucleic acid target locus of interest using said short RNA guide.

In general, the CRISPR/Cas or CRISPR system as used herein refers collectively to the elements involved in the expression of or directing the activity of CRISPR-associated (“Cas”) nuclease, including sequences encoding a Cas gene and one or more of, a tracr (transactivating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a“spacer” in the context of an endogenous CRISPR system), or “RNA(s)” as that term is herein used (e.g., RNA(s) to guide Cas, such as Cas9, e.g. CRISPR RNA and, where applicable, transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex.

In certain embodiments, the gRNA is a chimeric guide RNA or single guide RNA (sgRNA). In certain embodiments, the gRNA comprises a guide sequence and a tracr mate sequence (or direct repeat). In certain embodiments, the gRNA comprises a guide sequence, a tracr mate sequence (or direct repeat), and a tracr sequence. In certain embodiments, the CRISPR/Cas system or complex as described herein does not comprise and/or does not rely on the presence of a tracr sequence (e.g. if the Cas nuclease is Cas12a).

The CRISPR-Cas nuclease can be any such nuclease known in the art, such as a Cas9, Cas12a, Cas12b, Cas12i, Cas13a (formerly referred to as C2c2), C2c3, Cas13b or a modified version of any of the foregoing. CRISPR-Cas nucleases are well known in the art (see, e.g., Dong et al. Efficient Targeted Mutagenesis Mediated by CRISPR-Cas12a Ribonucleoprotein Complexes in Maize. Front. Genome Ed. (2021), vol. 3, article 670529; Wei et al. TALEN or Cas9—Rapid, Efficient and Specific Choices for Genome Modifications. J. of Genetics and Genomics (2013), vol. 40, pp. 281-289; Sedeek et al. Plant Genome Engineering for Targeted Improvement of Crop Traits. Frontiers in Plant Science (2019), vol. 10, article 114; and Zhang et al. Applications and potential of genome editing in crop improvement. Genome Biology (2018), vol. 19, article 210).

### EXAMPLES

### Example 1: Constructs Synthesized

Binary vector constructs were constructed containing differing combinations of transcriptional enhancers, promoters, transit peptides, and terminators, and variants of these genetic elements, driving expression of variants of eCry1Gb.1Ig. These genetic elements were synthesized and ligated into each binary vector through a restriction enzyme based cloning method. All promoters used were medium or strong constitutive promoters or viral promoters. Versions of eCry1Gb.1Ig genes were created with differing codon preferences to test desired expression level and efficacy. Table 1 shows the constructs created and lists the genetic elements with each coding sequence (CDS). Table 2 describes each of the genetic elements named in Table 1.

TABLE 1
Composition of Binary Constructs

| Construct | Cassette | | Transit | | |
|---|---|---|---|---|---|
| ID | position | Promoter | Peptide | CDS | Terminator |
| 24795 | 1 | prSoUbi4-02 | | eCry1Gb.1Ig-03 | tZmUbi361-05 |
| | 2 | prUbi1-43 | | cPMI-15 | tUbi1-04 |
| 23698 | 1 | prUbi1-18 | | eCry1Gb.1Ig-01 | tZmUbi361-01 |
| | 2 | prUbi1-18 | | cPMI-01 | tUbi1-04 |
| 24530 | 1 | prScBv-05 | | eCry1Gb.1Ig-02 | tNOS-05-01 |
| | 2 | prUbi1-18 | | cPMI-01 | tUbi1-04 |
| 24534 | 1 | pr35S-12 | | eCry1Gb.1Ig-02 | tNOS-05-01 |
| | 2 | prUbi1-43 | | cPMI-15 | tUbi1-04 |
| 25628 | 1 | prSoUbi4-02 | xOTPSSUct- | eCry1Gb.1Ig-03 | tZmUbi361-05 |
| | | | 02 | | |
| | 2 | prUbi1-43 | | cPMI-15 | tUbi1-04 |

TABLE 2
Description of Genetic Elements

| Element | Name | Description with relevant references |
|---|---|---|
| promoter | SoUbi4-02 | Constitutive Saccharum officinarum Ubiquitin 4 |
| | | promoter containing the first intron (NCBI accession |
| | | number AF093504.1). |
| promoter | ScBv-05 | Modified promoter from sugarcane Bacilliform IM |
| | | Badnavirus isolate Ireng Maleng (ScBVIM) (Davies et. |
| | | al. 2014). |
| promoter | Ubi1-18 | Promoter region from Zea mays polyubiquitin gene |
| | | which contains the first intron (NCBI accession number |
| | | S94464.1). Provides constitutive expression in monocots |
| | | (Christensen et al. 1992). |
| promoter | Ubi1-43 | Promoter region from Zea mays polyubiquitin gene |
| | | containing the first intron (NCBI accession number |
| | | S94464.1). Provides constitutive expression in monocots |
| | | (Christensen et al. 1992). |
| promoter | 35S-12 | Modified promoter from cauliflower mosaic virus (Odell |
| | | et al. 1985, Nature 313: 810-812). |
| transit | xOTPSSUct-02 | Optimized chimeric sunflower and maize rubisco small |
| peptide | | subunit transit peptide similar to pDPG434 (U.S. Pat. No. |
| | | 6,040,497). |
| coding | eCry1Gb.1Ig-01 | Codon-optimized gene encoding the engineered protein |
| sequence | | eCry1Gb.1Ig, which is a chimera of Cry1Gb and Cry1Ig. |
| | | Both Cry1Gb and Cry1Ig proteins are derived from |
| | | sequenced genomes of the soil bacterium Bacillus |
| | | thuringiensis and are active against several lepidopteran |
| | | pest species. The eCry1Gb.1Ig protein was engineered |
| | | to have improved insecticidal activity against fall |
| | | armyworm (Spodoptera frugiperda) (see, e.g., |
| | | International Patent Publication Number |
| | | WO2018111553). The eCry1Gb.1Ig protein also has |
| | | activity against other pest species including, e.g., oriental |
| | | armyworm (Mythimna separata), common |
| | | cutworm/oriental leafworm (Spodoptera litura), and |
| | | Asian corn borer (Ostrinia furnacalis) (see, e.g., |
| | | International Patent Application Number |
| | | PCT/CN2021/073190). |
| coding | eCry1Gb.1Ig-02 | Codon-optimized gene encoding the engineered protein |
| sequence | | eCry1Gb.1Ig, which is a chimera of Cry1Gb and Cry1Ig. |
| | | This version encodes the same protein but differs from |
| | | eCry1Gb.1Ig-01 by four base pairs to remove unintended |
| | | open reading frames. |
| coding | eCry1Gb.1Ig-03 | Sequence encoding the engineered protein eCry1Gb.1Ig, |
| sequence | | which is a chimera of Cry1Gb and Cry1Ig. Silent |
| | | mutations were introduced to optimize codon usage. |
| coding | PMI-01 | Escherichia coli gene pmi encoding the enzyme |
| sequence | | phosphomannose isomerase (PMI) (NCBI accession |
| | | number M15380.1); this gene is also known as manA. |
| | | Catalyzes the isomerization of mannose-6-phosphate to |
| | | fructose-6-phosphate (Negrotto et al. 2000). |
| coding | PMI-15 | Escherichia coli gene pmi encoding the enzyme |
| sequence | | phosphomannose isomerase (PMI) (NCBI accession |
| | | umber M15380.1); this gene is also known as manA. |
| | | Catalyzes the isomerization of mannose-6-phosphate to |
| | | fructose-6-phosphate (Negrotto et al. 2000). Silent |
| | | mutations were introduced as compared to PMI-01. |
| terminator | ZmUbi361-01 | Terminator derived from the maize Ubiquitin gene |
| | | (Nuccio 2018). |
| terminator | ZmUbi361-05 | Terminator derived from the maize Ubiquitin gene |
| | | (Nuccio 2018) with mutations to removed unintended |
| | | open reading frames compared to ZmUbi361-01 |
| | | terminator. |
| terminator | NOS-05-01 | Terminator sequence from the nopaline synthase (NOS) |
| | | gene of A. tumefaciens (NCBI accession number |
| | | V00087.1). Provides a polyadenylation site (Bevan et al. |
| | | 1983). |
| terminator | Ubi1-04 | Terminator from the ubiquitin 1 gene from Z. mays. One |
| | | bp mutation to remove an internal restriction site |
| | | compared to Ubi1-01 terminator. |

### Example 2: Agrobacterium-Mediated Transformation with Phosphomannose Isomerase (PMI) Selection

Each of the binary vector constructs was used to create maize transgenic events. Transformation of Zea mays to produce genetically modified maize was accomplished using immature embryos via Agrobacterium tumefaciens-mediated transformation, as described in Zhong et al. (2018) (Advances in Agrobacterium-mediated Maize Transformation. In: Lagrimini L. (eds) Maize. Methods in Molecular Biology, vol 1676. Humana Press, New York, NY). A. tumefaciens strain LBA4404 (recA−) harboring disarmed pTi plasmid pAL4404 and helper plasmid pVGW7 was used for maize transformation. Detailed information about the pAL4404 and pVGW7 plasmids is described by Hoekema et al. (Nature. (1983) 303:179-189), Ishida et al. (Nat Biotechnol (1996) 14:745-750) and Imayama et al. (U.S. Ser. No. 10/266,835). A. tumefaciens strain LBA4404 (recA⁻) containing individual binary vectors was prepared as described by Li et al. (Plant Physiol (2003) 133:736-47). For maize transformation, immature embryos from greenhouse grown maize inbred line NP2222 were harvested approximately 9 days after pollination and used as explants (Zhong et al., 2018). Immature embryo isolation, Agrobacterium inoculation and co-cultivation of Agrobacterium with the immature embryos were performed as described in Zhong et al. (2018) using the bulk extraction method described therein. Using this method, genetic elements within the left and right border regions of the transformation plasmid were efficiently transferred and integrated into the genome of the plant cell, while genetic elements outside these border regions were not transferred.

Transformed tissues and putative transgenic events were regenerated and rooted as described earlier (Zhong et al., 2018) using media with mannose selection for events containing a phosphomannose isomerase (PMI) selectable marker (Negrotto et al., (2000) Plant Cell Rep. 19:789-803.) or using 2 mM N-(Phosphonomethyl)-Glycine (TouchDown®) herbicide as the selection agent for events containing a modified version of a 5-enol pyruvylshikimate-3-phosphate synthase (EPSPS) enzyme.

Regenerated plantlets were tested for the presence of the target genes and plant selectable marker gene (PMI or EPSPS) by real-time TAQMAN® PCR analysis developed by Ingham et al. (Biotechniques 31(1):132-4, 136-40, 2001). Plants positive for target genes and selectable marker, also referred to as events, were transferred to the greenhouse for further propagation. In one plant transformed with binary vector 24795 (SEQ ID NO: 2), the expression cassette (SEQ ID NO:1) was found to contain a silent mutation in the coding sequence of cPMI-15 (SEQ ID NO: 7), creating a slightly modified expression cassette sequence in the plant (SEQ ID NO: 8). Upon further sequencing, additional mutations were found as shown in Table 3 (see also SEQ ID NOs: 9-31). The plants from which the sequencing results were obtained did not appear to have any significant negative impact on efficacy relative to the pool of other plants containing SEQ ID NO: 1.

TABLE 3
Additional variants identified by sequencing

| Position | | | | |
|---|---|---|---|---|
| in SEQ ID | | | | |
| NO 1 | Reference | Variant | Reference Sequence | Variant sequence |
| 324 | TC | T | CTCCCTCCTCCCCCGTTA | CTCCCTCCTCCCCGTTA (SEQ |
| | | | (SEQ ID NO: 32) | ID NO: 33) |
| 711 | G | T | TGATTCTGCGGGTTGGC | TGATTCTGCTGGTTGGC |
| | | | (SEQ ID NO: 34) | (SEQ ID NO: 35) |
| 7271 | A | AC | TAATAAATAGACACCCCC | TAATAAATAGACACCCCCCT |
| | | | TCCACACCCTCTT (SEQ | CCACACCCTCTT (SEQ ID |
| | | | ID NO: 36) | NO: 37) |
| 7387 | T | TC | CTCGTCCTCCCCCCCCCC | CTCGTCCTCCCCCCCCCCCCC |
| | | | CCCTC (SEQ ID NO: 38) | CTC (SEQ ID NO: 39) |
| 7989 | A | AT | CGGTCGTTCATTCGTTCT | CGGTCGTTCATTTCGTTCTA |
| | | | A (SEQ ID NO: 40) | (SEQ ID NO: 41) |
| 6015 | C | CT | AGACTAGTGGCTTGCTTT | AGACTAGTGGCTTGCTTTTT |
| | | | TTCGTATGTCT (SEQ ID | TCGTATGTCT (SEQ ID NO: |
| | | | NO: 42) | 43) |
| 6470 | AT | A | AAAAAATTACCACATATT | AAAAAATTACCACATATTTTT |
| | | | TTTTTTGTCACA (SEQ ID | TTGTCACA (SEQ ID NO: 45) |
| | | | NO: 44) | |
| 6683 | CT | C | TAGTGTGCATGTGTTCTC | TAGTGTGCATGTGTTCTCCT |
| | | | CTTTTTTTTTGCAAA (SEQ | TTTTTTTGCAAA (SEQ ID |
| | | | ID NO: 46) | NO: 47) |
| 7387 | T | TC | GTACGCCGCTCGTCCTCC | GTACGCCGCTCGTCCTCCCC |
| | | | CCCCCCCCCCCTCT (SEQ | CCCCCCCCCCTCT (SEQ ID |
| | | | ID NO: 48) | NO: 49) |
| 8397 | C | CA | GATCTCCGATCATGCAAA | GATCTCCGATCATGCAAAAA |
| | | | AACTCATTAACTCAGT | ACTCATTAACTCAGT (SEQ |
| | | | (SEQ ID NO: 50) | ID NO: 51) |
| 5944 | CT | C | CTTATGCAGAACCTTTTT | CTTATGCAGAACCTTTTTTTT |
| | | | TTTTG (SEQ ID NO: 52) | G (SEQ ID NO: 53) |
| 6015 | CT | C | GGAGACTAGTGGCTTGC | GGAGACTAGTGGCTTGCTTT |
| | | | TTTTTCGTATGTCT (SEQ | TCGTATGTCT (SEQ ID NO: |
| | | | ID NO: 54) | 55) |
| 7387 | T | TC | ACGCCGCTCGTCCTCCCC | ACGCCGCTCGTCCTCCCCCC |
| | | | CCCCCCCCCTCT (SEQ ID | CCCCCCCCTCT (SEQ ID NO: |
| | | | NO: 56) | 57) |
| 7576 | TG | T | GCCAGTGTTTCTCTTTGG | GCCAGTGTTTCTCTTTGGGA |
| | | | GGAATCCTGGGAT (SEQ | ATCCTGGGAT (SEQ ID NO: |
| | | | ID NO: 58) | 59) |
| 10055 | G | GT | ACTAACAATTAGTTTCAG | ACTAACAATTAGTTTTCAGT |
| | | | TGCATTCAAACA (SEQ ID | GCATTCAAACA (SEQ ID NO: |
| | | | NO: 60) | 61) |
| 347 | TC | T | TTATAAATTGGCTTCATC | TTATAAATTGGCTTCATCCCT |
| | | | CCCTCCTTGCCTCAT (SEQ | CCTTGCCTCAT (SEQ ID NO: |
| | | | ID NO: 62) | 63) |
| 1579 | A | AT | CATATATCATGTATTTTTT | CATATATCATGTATTTTTTTT |
| | | | TTTGG (SEQ ID NO: 64) | TTGG (SEQ ID NO: 65) |
| 7387 | T | TC | TACGCCGCTCGTCCTCCC | TACGCCGCTCGTCCTCCCCC |
| | | | CCCCCCCCCCT (SEQ ID | CCCCCCCCCT (SEQ ID NO: |
| | | | NO: 66) | 67) |
| 7720 | CT | C | CATCTTTTCATGCTTTTTT | CATCTTTTCATGCTTTTTTTG |
| | | | TTGTCTTGGTTGTGATG | TCTTGGTTGTGATG (SEQ ID |
| | | | (SEQ ID NO: 68) | NO: 69) |
| 8656 | A | G | CCTGTTCAAAGTATTATG | CCTGTTCAAAGTATTGTGCG |
| | | | CGCAGCACAGCCA (SEQ | CAGCACAGCCA (SEQ ID NO: |
| | | | ID NO: 70) | 71) |
| 8870 | GC | G | GTCTCCCTACTCCAGCCG | GTCTCCCTACTCCAGCGGTC |
| | | | GTCGCAGGTGCAC (SEQ | GCAGGTGCAC (SEQ ID NO: |
| | | | ID NO: 72) | 73) |
| 9064 | AC | A | AATTTCTGAATTTTACCC | AATTTCTGAATTTTACCGGA |
| | | | GGAAGACAGCGG (SEQ | AGACAGCGG (SEQ ID NO: |
| | | | ID NO: 74) | 75) |

### Example 3: Quantitative ELISA for Detection of Trait Proteins

Detection of the different trait proteins used two monoclonal antibodies produced against each protein. Samples were taken from the leaves of transgenic events and extracted in phosphate buffered saline pH 7.3 (PBS) containing 0.05% Tween-20 (PBST). Total soluble protein (TSP) of the extract was measured using the Pierce BCA Protein Assay (Thermo Scientific, Rockford, IL). High-binding polystyrene plates (Nunc Maxisorp #430341) were coated at 4° C. overnight with 1 sg/ml of the specific monoclonal antibody (MAb) in 25 mM borate, 75 mM NaCl, pH 8.5. Plates were washed five times with PBST. Samples or standards in ELISA diluent (PBST containing 1% bovine serum albumin) were added to the plate (100 μl/well), incubated for 1 hr at room temperature (RT) with shaking, and washed five times. HRP-labeled secondary MAb (100 μl/well) diluted 1/10,000 in ELISA diluent was then added to the plate, incubated for 1 hr at ambient temperature with shaking, and washed as before. Substrate Tetramethylbenzidine (SurModics, Eden Prairie, MN) was added (100 μl/well) and allowed to develop for 15-30 min at room temperature with shaking. The reaction was stopped using 1 N HCl (100 μl/well). The absorbance was measured at 450 nm using a microplate reader (BioTek Powerwave XS2, Winooski, VT). The standard curve used a four-parameter curve fit to plot the concentrations versus the absorbance. To normalize for extraction efficiency, the concentration of each analyte was divided by the concentration of the total soluble protein (TSP).

TABLE 4
Summary of ELISA expression data

| Construct | # of | ng eCry1Gb.1Ig /mg | ng eCry1Gb.1Ig/mg |
|---|---|---|---|
| ID | events | TSP (average) | TSP (range) |
| 24795 | 257 | 23 | 1-121 |
| 23698 | 43 | 57 | 30-125 |
| 24530 | 13 | 0.4 | 0.1-0.8 |
| 24534 | 29 | 0.9 | 0.3-2.3 |
| 25628 | 16 | 0 | NA |

Constructs 24530, 24534 and 25628 surprisingly only produced events with very low or no expression of the trait protein, even though the trait protein sequence was paired with a promoter that was expected to be a medium or strong promoter.

### Example 4: Greenhouse Efficacy Testing

279 transgenic corn events from construct 24795 were confirmed to have single copy tDNA insertion and expression of trait protein via ELISA analysis as described in Example 3.

From this population, 45 transgenic corn events from construct 24795 as well transgenic corn events from the other constructs mentioned in Table 4 were selected for bioassay testing. The events selected represented a range of eCry1Gb.1Ig expression, comprising a mixture of low, medium, and high expressors. The bioassay sampling consisted of a detached leaf bioassay, where a portion of the leaf was excised from the plant, placed into a petri dish with a sterile water moistened filter pad, and infested with approximately 10 fall armyworm (Spodoptera frugiperda) neonate larvae. The assays were incubated at ambient laboratory temperature and scored 5 days after infestation. Each sample was scored for percentage of leaf protection (scale 1-5) and insect mortality (scale 1-3). Events which received a percentage of leaf protection rating of 1 or 2 (i.e., less than 5% damage to the excised leaf disk) and achieved 100% mortality of the neonate larvae were considered efficacious and used as a benchmark for the construct's performance. The bioassay data for the 45 events tested was extrapolated to those events whose trait gene expression was similar, resulting in a total of 65 24795 events meeting the efficacy and expression criteria and progressing for further characterization. Events from constructs 23698, 24530, 24534, and 25628 did not meet the efficacy and expression criteria; those constructs were not selected for further progression.

### Example 5: Field Efficacy Trials

24 transgenic corn events from construct 24795 were tested in field cycles in Argentina. Events were planted in one plot rows with 3 replications each. Ratings for foliar fall armyworm (Spodoptera frugiperda) were assessed from eight plants for each row. The foliar leaf damage was evaluated using the Davis Scale of 0-9, (Davis, F. M. & Williams, W. P. 1992. Visual rating scales for screening whorl-stage corn for resistance to fall armyworm. Mississippi Agricultural & Forestry Experiment Station, Technical Bulletin 186, Mississippi State University, MS39762, USA.). 14 of 24 events from the above construct had acceptable efficacy against fall armyworm.

1. A recombinant nucleic acid molecule comprising a nucleic acid sequence that is at least 99% identical to any of SEQ ID NO: 1, 3, or 8 to 31, or the complement thereof, wherein the nucleic acid sequence encodes a polypeptide comprising the sequence of SEQ ID NO: 4.

2-5. (canceled)

6. A transgenic host cell comprising the nucleic acid molecule of claim 1.

7. The transgenic host cell of claim 6, wherein the cell is a bacterial cell or a plant cell.

8. The transgenic host cell of claim 7, wherein the cell is a bacterial cell and the bacterial cell is an Escherichia coli, Bacillus thuringiensis, Bacillus subtilis, Bacillus megaterium, Bacillus cereus, Agrobacterium ssp. or Pseudomonas ssp. cell.

9. The transgenic host cell of claim 7, wherein the cell is a plant cell and the plant cell is a maize, sorghum, wheat, sunflower, tomato, crucifers, oat, turf grass, pasture grass, peppers, potato, cotton, rice, soybean, sugarcane, sugar beet, tobacco, barley, or oilseed rape cell.

10. The transgenic host cell of claim 9, wherein the plant cell is a maize cell.

11. A transgenic plant or plant part comprising the nucleic acid molecule of claim 1.

12. The transgenic plant of claim 11, wherein the plant is a monocot plant.

13. The transgenic plant of claim 11, wherein the plant is a dicot plant.

14. The transgenic plant of claim 11, wherein;
a) the plant is selected from the group consisting of maize, sorghum, wheat, sunflower, tomato, crucifers, oat, turf grass, pasture grass, peppers, potato, cotton, rice, soybean, sugarcane, sugar beet, tobacco, barley, and oilseed rape, or
b) the plant part is a seed.

15. (canceled)

16. A progeny or propagule of any generation of the plant of claim 11, wherein the progeny or propagule comprises the nucleic acid molecule.

17-19. (canceled)

20. A method of producing a transgenic plant with enhanced insecticidal properties, comprising introducing the nucleic acid molecule of claim 1 into a plant thereby producing a transgenic plant, wherein the nucleic acid molecule expresses effective insect-controlling amounts of protein.

21. A method of producing a transgenic plant with enhanced insecticidal properties, comprising the steps of:
a) providing the nucleic acid molecule of claim 1;
b) introducing into a plant, tissue culture, or a plant cell the nucleic acid molecule of step (a) to obtain a transformed plant, transformed tissue culture, or a transformed cell having enhanced insecticidal properties; and
c) growing the transformed plant or regenerating a transformed plant from the transformed tissue culture or transformed plant cell, so a transgenic plant with enhanced insecticidal properties is produced.

22. A method of producing transgenic seed, comprising the steps of:
a) obtaining a fertile transgenic plant of claim 11; and
b) growing the plant under appropriate conditions to produce the transgenic seed.

23. A method of producing progeny of any generation of a fertile transgenic plant with enhanced insecticidal properties, comprising the steps of:
a) obtaining a fertile transgenic plant with enhanced insecticidal properties comprising the nucleic acid molecule of claim claim 1;
b) collecting transgenic seed from the transgenic plant;
c) planting the collected transgenic seed; and
d) growing the progeny transgenic plants from the seed,
wherein the progeny has enhanced insecticidal properties relative to a non-transformed plant.

24. A method for producing a transgenic plant with enhanced insecticidal properties, comprising the steps of sexually crossing a first parent plant with a second parent plant, wherein the first or second parent plant is the plant of claim 11, to produce a first generation progeny plant that comprises the nucleic acid molecule.

25. A method for producing a transgenic plant with enhanced insecticidal properties, comprising the steps of:
a) sexually crossing a first parent plant with a second parent plant, wherein the first or second parent plant is the plant of claim 11; and
b) selecting a first generation progeny plant with enhanced insecticidal properties, wherein the selected progeny plant comprises the nucleic acid molecule.

26. The method of claim 25, further comprising the steps of:
a) selfing the first generation progeny plant, thereby producing a plurality of second generation progeny plants; and
b) selecting from the second generation progeny plants a plant with enhanced insecticidal properties, wherein the selected second generation progeny plants comprise the nucleic acid molecule.

27. A method of controlling a lepidopteran pest comprising feeding the pest a plant or plant part comprising the nucleic acid molecule of claim 1.

28. The method of claim 27, wherein the lepidopteran pest is a Spodoptera frugiperda (fall armyworm) pest.

29. A method of producing a commodity plant product, the method comprising using the plant of any one of claim 11 to produce said commodity plant product therefrom.

30. The method of claim 29, wherein the commodity plant product is a grain, starch, seed oil, syrup, flour, meal, starch, cereal, or protein.

31. A method of detecting the presence of a nucleic acid molecule in a sample, the method comprising:
(a) contacting the sample with a pair of primers that, when used in a nucleic-acid amplification reaction with DNA comprising the nucleic acid molecule of claim 1, produces an amplicon that is diagnostic for the nucleic acid molecule;
(b) performing a nucleic acid amplification reaction, thereby producing the amplicon; and
(c) detecting the amplicon.

32. A method of detecting the presence of a nucleic acid molecule in a sample, the method comprising:
(a) contacting the sample with a probe that hybridizes under high stringency conditions with DNA comprising the nucleic acid molecule of claim 1 and does not hybridize under high stringency conditions with DNA of a control maize plant not comprising the nucleic acid molecule;
(b) subjecting the sample and probe to high stringency hybridization conditions; and
(c) detecting hybridization of the probe to the nucleic acid molecule.

33. A pair of polynucleotide primers comprising a first polynucleotide primer and a second polynucleotide primer which function together in the presence of the nucleic acid molecule of claim 1 in a sample to produce an amplicon diagnostic for the presence of the nucleic acid molecule in a sample.

34. The pair of polynucleotide primers of claim 33, wherein the first polynucleotide primer comprises at least 10 contiguous nucleotides that are complementary to any one of SEQ ID NOs: 1 or 8 to 31 and the second polynucleotide primer comprises at least 10 contiguous nucleotides that are complementary to the reverse complement of any one of SEQ ID NOs: 1 or 8 to 31.

35. A kit for detecting the nucleic acid molecule of claim 1, the kit comprising at least one nucleic acid molecule of sufficient length of contiguous nucleotides to function as a primer or probe in a nucleic acid detection method, and which upon amplification of or hybridization to a target nucleic acid sequence in a sample followed by detection of the amplicon or hybridization to the target sequence, are diagnostic for the presence of the nucleic acid molecule.

36. The kit of claim 35, wherein the at least one nucleic acid molecule comprises at least 10 contiguous nucleotides that are complementary to any one of SEQ ID NOs: 1 or 8 to 31.

37. A method, comprising introducing a modification into the nucleic acid molecule present in a transgenic host cell of any one of claims 6 to 10 or a transgenic plant of claim 11, thereby producing a modified transgenic host cell or a modified transgenic plant.

38. The method of claim 37, wherein the modification is a deletion, an insertion, a substitution, a duplication, or inversion or a combination thereof.

39. The method of claim 38, wherein the modification comprises deletion of a portion or all of a selectable marker coding sequence present in the nucleic acid molecule.

40. The method of claim 37, wherein the modification is introduced using a nuclease or homologous recombination, or a combination thereof.

41. The method of claim 40, wherein the nuclease is a CRISPR-Cas nuclease.

42. The method of claim 37, wherein the method further comprises producing a plant from the modified transgenic host cell and selfing or crossing the plant with another plant, thereby producing a modified transgenic progeny plant.

43. The method of claim 37, wherein the method further comprises selfing or crossing the modified transgenic plant with another plant, thereby producing a modified transgenic progeny plant.

44. The method of claim 42, wherein the method further comprises selfing or outcrossing the modified transgenic progeny plant for at least one additional generation.
